(19) United States
(12) Patent Application Publication
(10) Pub. No.: US 2006/0100225 A1
(43) Pub. Date: May 11, 2006

(54) Heteroaryl guanidines; inhibitors of viral replication

(76) Inventors: Xi Chen, Killingworth, CT (US); Ciuxan Liu, Branford, CT (US); Andrew Thurkauf, Ridgefield, CT (US); Shirley Louise-May, Guilford, CT (US)

Correspondence Address: CANTOR COLBURN, LLP, 55 GRIFFIN ROAD SOUTH, BLOOMFIELD, CT, 06002, US

(21) Appl. No.: 11/088,276

(22) Filed: Mar. 23, 2005

Related U.S. Application Data
(60) Provisional application No. 60/555,872, filed on Mar. 23, 2004.

(51) Int. Cl. A61K 31/513; A61K 31/505; C07D 239/32

(52) U.S. Cl. 514/269; 514/275; 544/320; 544/330

(57) ABSTRACT

Compounds of Formula 1, and pharmaceutically acceptable forms thereof, are provided: 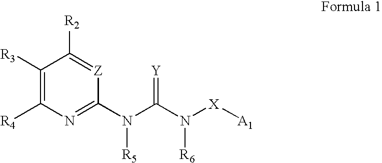
wherein the variables X, Y, Z, A₁, R₂, R₃, R₄, R₅ and R₆ are defined herein. Certain compounds of Formula 1 described herein which possess potent antiviral activity. Certain compounds of Formula 1 that are potent and/or selective inhibitors of Hepatitis C virus replication. Pharmaceutical compositions containing one or more compounds of Formula 1, or a salt, solvate, or acylated prodrug of such compounds, and one or more pharmaceutically acceptable carriers, excipients, or diluents are also provided. Methods of treating patients suffering from certain infectious diseases by administering to such patients an amount of a compound of Formula 1 effective to reduce signs or symptoms of the disease or disorder are disclosed. These infectious diseases include viral infections, particularly HCV infections. Methods of treating human patients suffering from an infectious disease, but also encompasses methods of treating other animals, including livestock and domesticated companion animals, suffering from an infectious disease. Methods of treatment include administering a compound of Formula 1 as a single active agent or administering a compound of Formula 1 in combination with on or more other therapeutic agent.

### CROSS REFERENCE TO RELATION APPLICATION

This application claims priority to U.S. provisional patent application No. 60/555,872 filed Mar. 23, 2004, which is hereby incorporated by reference in its entirety.

### FIELD OF THE INVENTION

Heteroaryl guanidine compounds, particularly useful as antiviral agents. Certain heteroaryl guanidine compounds disclosed herein are potent and/or selective inhibitors of viral replication, particularly Hepatitis C virus replication are provided herein. Pharmaceutical compositions containing one or more heteroaryl guanidine compounds and one or more pharmaceutically acceptable carriers, excipients, or diluents are also provided herein. Such pharmaceutical compositions may contain a heteroaryl guanidine compound as the only active agent or may contain a combination of a heteroaryl guanidine compound and one or more other pharmaceutically active agents. Methods for treating Hepatitis C viral infections in mammals are provided herein.

### BACKGROUND

In the 1940's the disease originally referred to as viral hepatitis was distinguished into two separate disorders termed infectious hepatitis (hepatitis A, HAV) and homologous serum hepatitis (hepatitis B, HBV). Transfusion of blood products had been demonstrated to be a common route of transmission of viral hepatitis. HBV was originally assumed to be the causative agent of post-transfusion hepatitis as the epidemiological and clinical features of the disorder did not fit those of HAV.

Soon after a radioimmunoassay for hepatitis B surface antigen (HBsAg) became available as a tool for identifying patients infected with HBV, it became apparent that most patients having post-transfusion hepatitis were negative for HBsAg. Thus, hepatitis following blood transfusion that was not caused by hepatitis A or hepatitis B and was subsequently referred to as non-A, non-B hepatitis.

The causative agent of non-A, non-B hepatitis (hepatitis C virus, HCV) was discovered in 1989 via screening of cDNA expression libraries made from RNA and DNA from chimpanzees infected with serum from a patient with post-transfusion non-A, non-B hepatitis. To identify portions of the genome that encoded viral proteins, the libraries were screened with antibodies from patients who had non-A, non-B hepatitis. These investigators went on to show that the virus they identified was responsible for the vast majority of cases of non-A, non-B hepatitis.

The hepatitis C virus is one of the most prevalent causes of chronic liver disease in the United States. It accounts for about 15 percent of acute viral hepatitis, 60 to 70 percent of chronic hepatitis, and up to 50 percent of cirrhosis, end-stage liver disease, and liver cancer. Almost 4 million Americans, or 1.8 percent of the U.S. population, have antibodies to HCV (anti-HCV), indicating ongoing or previous infection with the virus. Hepatitis C causes an estimated 8,000 to 10,000 deaths annually in the United States. Hepatitis C virus (HCV) infection occurs throughout the world, and, prior to its identification, represented the major cause of transfusion-associated hepatitis. The seroprevalence of anti-HCV in blood donors from around the world has been shown to vary between 0.02% and 1.23%. HCV is also a common cause of hepatitis in individuals exposed to blood products. There have been an estimated 150,000 new cases of HCV infection each year in the United States alone during the past decade. The acute phase of HCV infection is usually associated with mild symptoms. However, evidence suggests that only 15%-20% of the infected people will clear HCV. Among the group of chronically infected people, 10-20% will progress to life-threatening conditions known as cirrhosis and another 1-5% will develop a liver cancer called hepatocellular carcinoma. Unfortunately, the entire infected population is at risk for these life-threatening conditions because no one can predict which individual will eventually progress to any of them.

HCV is a small, enveloped, single-stranded positive RNA virus in the Flaviviridae family. The genome is approximately 10,000 nucleotides and encodes a single polyprotein of about 3,000 amino acids. The polyprotein is processed by host cell and viral proteases into three major structural proteins and several non-structural proteins necessary for viral replication. Several different genotypes of HCV with slightly different genomic sequences that correlate with differences in response to treatment with interferon alpha have since been identified.

HCV replicates in infected cells in the cytoplasm, in close association with the endoplasmic reticulum. Incoming positive sense RNA is released and translation is initiated via an internal initiation mechanism. Internal initiation is directed by a cis-acting RNA element at the 5′ end of the genome; some reports have suggested that full activity of this internal ribosome entry site, or IRES, is seen with the first 700 nucleotides, which spans the 5′ untranslated region (UTR) and the first 123 amino acids of the open reading frame (ORF). All the protein products of HCV are produced by proteolytic cleavage of a large (approximately 3000 amino acid) polyprotein, carried out by one of three proteases: the host signal peptidase, the viral self-cleaving metalloproteinase, NS2, or the viral serine protease NS3/4A. The combined action of these enzymes produces the structural proteins (C, E1 and E2) and non-structural (NS2, NS3, NS4A, NS4B, NS5A, and NS5B) proteins that are required for replication and packaging of viral genomic RNA. NS5B is the viral RNA-dependent RNA polymerase (RDRP) that is responsible for the conversion of the input genomic RNA into a negative stranded copy (complimentary RNA, or cRNA; the cRNA then serves as a template for transcription by NS5B of more positive sense genomic/messenger RNA.

An effective vaccine is greatly needed, yet development is unlikely in the near future because: i) lack of an efficient cell culture system and small animal models; ii) a weak neutralizing humoral and protective cellular immune response; iii) marked genetic variability of the virus, and iv) the lack of a viral proofreading mechanism.

Several institutions and laboratories are attempting to identify and develop anti-HCV drugs. Currently the only effective therapy against HCV is alpha-interferon, which reduces the amount of virus in the liver and blood (viral load) in only a small proportion of infected patients. Alpha interferon was first approved for use in HCV treatment more than ten years ago. Alpha interferon is a host protein that is made in response to viral infections and has natural antiviral activity. These standard forms of interferon, however, are now being replaced by pegylated interferons (peginterferons). Peginterferon is alpha interferon that has been modified chemically by the addition of a large inert molecule of polyethylene glycol. At the present time, the optimal regimen appears to be a 24- or 48-week course of the combination of pegylated alpha interferon and the nucleoside Ribavarin, an oral antiviral agent that has activity against a broad range of viruses. By itself, Ribavarin has little effect on HCV, but adding it to interferon increases the sustained response rate by two- to three-fold. Nonetheless, response rates to the combination interferon/Ribavarin therapy are moderate, in the range 50-60%, although response rates for selected genotypes of HCV (notably genotypes 2 and 3) are typically higher. Among patients who become HCV RNA negative during treatment, a significant proportion relapse when therapy is stopped.

In addition, there are often significant adverse side effects associated with each of these agents. Patients receiving interferon often present with flu-like symptoms. Pegylated interferon has been associated with bone marrow suppressive effects. Importantly, alpha interferon has multiple neuropsychiatric effects. Prolonged therapy can cause marked irritability, anxiety, personality changes, depression, and even suicide or acute psychosis. Interferon therapy has also been associated with relapse in people with a previous history of drug or alcohol abuse.

Side effects of Ribavarin treatment include histamine-like side effects (itching and nasal stuffiness) and anemia due to dose related hemolysis of red cells and histamine like side effects.

Taken together, the proceeding facts indicate a significant need for effective small molecule inhibitors of hepatitis C virus replication that do not suffer from the above-mentioned drawbacks.

### SUMMARY OF THE INVENTION

Compounds of Formula 1 (shown below) are provided herein. Formula I includes heteroaryl guanidines and related compounds, which possess antiviral activity. Other embodiments provide compounds of Formula 1 that are potent and/or selective inhibitors of Hepatitis C virus replication. Additionally pharmaceutical compositions containing one or more compound of Formula 1, or a salt, solvate, or acylated prodrug of such compounds, and one or more pharmaceutically acceptable carriers, excipients, or diluents are provided herein.

Methods of treating patients suffering from certain infectious diseases comprising administering to such patients an amount of a compound of Formula 1 effective to reduce signs or symptoms of the disease or disorder are also provided herein. These infectious diseases include viral infections, particularly HCV infections. The invention includes methods of treating human patients suffering from an infectious disease, but also encompasses methods of treating other animals, including livestock and domesticated companion animals, suffering from an infectious disease.

Methods of treatment include administering a compound of Formula 1 as a single active agent or administering a compound of Formula 1 in combination with one or more other therapeutic agent.

Thus a first embodiment provides compounds of Formula 1:

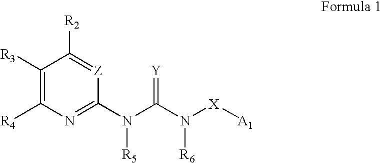

and the pharmaceutically acceptable forms thereof.

In Formula 1, the variables X, Y, Z, R₂-R₆, and A₁ carry the following definitions.

X is absent, —CR′R″—, —(CR′R″)₂—, —CR′R″O—, —O—, or NR.

R is hydrogen, C₁-C₆alkyl, or cyano; and R′ and R″ are independently hydrogen, halogen, C₁-C₂alkyl, or C₁-C₂alkoxy.

Y is —CHNO₂, —NSO₂H, —NSO₂(C₁-C₆alkyl), or NR.

Z is N or CR₁.

R₁, R₂, R₃, and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy).

Alternatively, R₁ and R₄ carry the definition set forth above and R₂ and R₃ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino.

Or, R₁ and R₂ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); and R₃ and R₄ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino.

R₅ and R₆ are independently hydrogen, C₁-C₄alkyl, C₂-C₄alkenyl, or C₃-C₈cycloalkyl(C₀-C₂alkyl).

A₁ is i) phenyl, which is substituted with 1 LA₂ substituent and 0 or 1 or more R₇ substituents; or ii) naphthyl, indanyl, or 9H-fluorenyl, each of which is substituted with 0 or 1 LA₂ substituent, and 0 or 1 or more R₇ substituents; or iii) heteroaryl, which is substituted with 0 or 1 LA₂ substituent, and 0 or 1 or more R₇ substituents.

R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy).

L is absent, —CR₈R₉—, —(CR₈R₉)₂—, —CR₈R₉O—, —OCR₈R₉—, —O(CR₈R₉)₂—, —NH(C═O)—, —NH(SO₂)—, —O—, or NR₁₀; where R₈ and R₉ are independently hydrogen, halogen, C₁-C₂alkyl, or C₁-C₂alkoxy; and R₁₀ is hydrogen, C₁-C₆alkyl, or cyano.

A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- and di-(C₁-C₆alkyl)amino, mono- and di-(C₁-C₄alkyl)amino(C₁-C₄alkyl), and mono- and di-(C₁-C₄alkyl)amino(C₁-C₄alkoxy).

Certain compounds of Formula 1 disclosed herein exhibit good activity in an HCV replication assay, such as the HCV replicon assay set forth in Example 3, which follows. Preferred compounds of Formula 1 exhibit an EC₅₀ of about 10 micromolar or less, or more preferably an EC₅₀ of about 1 micromolar or less; or still more preferably an EC₅₀ of about 500 nanomolar or less in an HCV replicon assay.

### DETAILED DESCRIPTION OF THE INVENTION

Chemical Description and Terminology

Prior to setting forth the invention in detail, it may be helpful to provide definitions of certain terms to be used herein. Compounds of the present invention are described using standard nomenclature. Unless defined otherwise, all technical and scientific terms used herein have the same meaning as is commonly understood by one of skill in the art to which this invention belongs.

Formula 1 includes all subformulae thereof. For example Formula 1 includes compounds of Formulae 2-12.

In certain situations, the compounds of Formula 1 may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present invention. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Where a compound exists in various tautomeric forms, the invention is not limited to any one of the specific tautomers, but rather includes all tautomeric forms.

The present invention is intended to include all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula that includes variables, e.g. X, Y, Z, R₂-R₆, and A₁. Unless otherwise specified, each variable within such a Formula 1 is defined independently of other variables. Thus, if a group is said to be substituted, e.g. with 0-2 R*, then said group may be substituted with up to two R* groups and R* at each occurrence is selected independently from the definition of R*. Also, combinations of substituents and/or variables are permissible only if such combinations result in stable compounds.

The term “substituted”, as used herein, means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified substituents described herein are “named into the ring.” Thus the point of attachment of the substituent “phenylalkyl” to a ring system is on the alkyl portion rather than the phenyl portion of the substituent.

The phrase “optionally substituted” indicates that such groups may either be unsubstituted or substituted at one or more of any of the available positions, typically 1, 2, 3, or 4 positions, by one or more suitable groups such as those disclosed herein.

Suitable groups that may be present on a “substituted” position include, but are not limited to, e.g., halogen; cyano; hydroxyl; nitro; azido; alkanoyl (such as a C₂-C₆ alkanoyl group such as acyl or the like); carboxamido; alkyl groups (including cycloalkyl groups, having 1 to about 8 carbon atoms, or 1 to about 6 carbon atoms); alkenyl and alkynyl groups (including groups having one or more unsaturated linkages and from 2 to about 8, or 2 to about 6 carbon atoms); alkoxy groups having one or more oxygen linkages and from 1 to about 8, or from 1 to about 6 carbon atoms; aryloxy such as phenoxy, napthyloxy, and 5,6,7,8-tetrahydronapthyloxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups, which may have a single nitrogen atom or more than one nitrogen atoms, and from 1 to about 8, or from 1 to about 6 carbon atoms; aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being an exemplary arylalkyl group; arylalkoxy having 1 to 3 separate or fused rings and from 6 to about 18 ring carbon atoms, with benzyloxy being an exemplary arylalkoxy group; or a saturated, unsaturated, or aromatic heterocyclic group having 1 to 3 separate or fused rings with 3 to about 8 members per ring and at least one ring having one or more N, O, or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino. Heterocyclic groups include, for example, bicyclic groups in which one ring is a heterocyclic ring and the other ring is a carbocyclic ring, such as a benzene ring, e.g., a benzothiazolyl benzofuranyl group.

“Adjacent atoms” are atoms connected by a covalent bond. For example adjacent carbon atoms may be carbon atoms at consecutive positions in an aromatic ring connected to each other with an aromatic bond.

A dash (“-”) that is not between two letters or symbols is used to indicate a point of attachment for a substituent. For example, —SH is attached through sulfur of the mercapto group.

As used herein, “acetyl” is a group of the formula —(C═O)CH₃.

As used herein, “alkyl” includes both branched and straight chain saturated aliphatic hydrocarbon groups, having the specified number of carbon atoms, generally from 1 to about 12 carbon atoms. The term C₁-C₆alkyl as used herein indicates an alkyl group having from 1 to about 6 carbon atoms. When C₀-Cₙ alkyl is used herein in conjunction with another group, for example, cycloalkyl(C₀-C₄ alkyl), the indicated group, in this case cycloalkyl, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, 3-methylbutyl, t-butyl, n-pentyl, and sec-pentyl. Alkyl groups described herein typically have from 1 to about 12 carbons atoms. Preferred alkyl groups are lower alkyl groups, those alkyl groups having from 1 to about 8 carbon atoms, from 1 to about 6 carbon atoms, or from 1 to about 4 carbons atoms e.g. C₁-C₈, C₁-C₆, and C₁-C₄ alkyl groups.

“Alkenyl” as used herein, indicates a straight or branched hydrocarbon chain comprising one or more carbon-carbon double bonds, which may occur in any stable point along the chain. Alkenyl groups described herein typically have from 2 to about 12 carbons atoms. Preferred alkenyl groups are lower alkenyl groups, those alkenyl groups having from 2 to about 8 carbon atoms, e.g. C₂-C₈, C₂-C₆, and C₂-C₄ alkenyl groups. Examples of alkenyl groups include ethenyl, propenyl, and butenyl groups.

“Alkynyl” as used herein, indicates a straight or branched hydrocarbon chain comprising one or more carbon-carbon triple bonds that may occur in any stable point along the chain, such as ethynyl and propynyl. Alkynyl groups described herein typically have from 2 to about 12 carbons atoms. Preferred alkynyl groups are lower alkynyl groups, those alkynyl groups having from 2 to about 8 carbon atoms, e.g. C₂-C₈, C₂-C₆, and C₂-C₄ alkynyl groups.

“Alkoxy” indicates an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkoxy(C₁-Cₙalkyl) indicates an alkoxy group as defined herein with the indicated number of carbon atoms attached through its oxygen bridge to an alkyl group as described herein having from 1 to n carbon atoms and attached via a single covalent bond to the group it substitutes.

“Alkoxy(C₁-Cₙalkoxy) indicates an alkoxy group as defined herein with the indicated number of carbon atoms attached through its oxygen bridge to a second alkoxy group as described herein having from 1 to n carbon atoms and attached via an oxygen bridge to the group it substitutes.

“Alkoxy(C₁-Cₙalkylamino) indicates an alkoxy group as defined herein with the indicated number of carbon atoms attached through its oxygen bridge to an alkylamino group as described herein, the alkyl portion having from 1 to n carbon atoms and attached via an amino bridge to the group it substitutes.

“Alkanoyl” indicates an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

As used herein, “alkylthio” means alkyl-S—, where the alkyl group is an alkyl group as defined above having the defined number of carbon atoms. An exemplary alkylthio group is methylthio.

As used herein “aminoalkyl” is an alkyl group as defined herein, having the indicated number of carbon atoms, and substituted with at least one amino substituent (—NH₂). When indicated, aminoalkyl groups, like other groups described herein, may be additionally substituted.

As used herein, the term “mono- and/ or di-alkylamino” indicates secondary or tertiary alkyl amino groups, wherein the alkyl groups are as defined above and have the indicated number of carbon atoms. The point of attachment of the alkylamino group is on the nitrogen. The alkyl groups are independently chosen. Examples of mono- and di-alkylamino groups include ethylamino, dimethylamino, and methyl-propyl-amino. “Mono- and/or dialkylaminoalkyl” groups are mono- and/ or di-alkylamino groups attached through an alkyl linker having the specified number of carbon atoms, for example a di-methylaminoethyl group. Tertiary amino substituents may by designated by nomenclature of the form N—R—N—R′, indicating that the groups R and R′ are both attached to a single nitrogen atom.

As used herein, the term “mono- and/ or di-alkylaminoalkoxy” indicates mono- and/or di-alkylamino groups as described above attached through an alkoxy linker having the specified number of carbon atoms.

As used herein, the term “aryl” indicates aromatic groups containing only carbon in the aromatic ring or rings. Such aromatic groups may be further substituted with carbon or non-carbon atoms or groups. Typical aryl groups contain 1 or 2 separate, fused, or pendant rings and from 6 to about 12 ring atoms, without heteroatoms as ring members. Where indicated aryl groups may be substituted. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl.

The term “carbocyclic group” indicates a 3 to 8 membered saturated, partially unsaturated, or aromatic ring containing only carbon ring atoms or a 6-11 membered saturated, partially unsaturated, or aromatic bicyclic ring system containing only carbon ring atoms. Unless otherwise indicated, the carbocyclic group may be attached to its pendant group at any carbon atom that results in a stable structure. In certain instances herein 5- to 6-membered carbocyclic groups are preferred. When indicated the carbocyclic groups described herein may be substituted on any available ring carbon if the resulting compound is stable. Carbocyclic groups include, cycloalkyl groups, such as cyclopropyl, cyclohexyl, cycloalkenyl groups, such as cycloalkenyl, bridged cycloalkyl groups, and aryl groups, such as phenyl.

“Cycloalkyl” as used herein, indicates a monocyclic or multicyclic saturated hydrocarbon ring group, having the specified number of carbon atoms, usually from 3 to about 10 ring carbon atoms. Monocyclic cycloalkyl groups typically have from 3 to about 8 carbon ring atoms or from 3 to about 7 carbon ring atoms. Multicyclic cycloalkyl groups may have 2 or 3 fused cycloalkyl rings or contain bridged or caged cycloalkyl groups. Cycloalkyl substituents may be pendant to a substituted nitrogen or carbon atom, or when bound to a substituted carbon atom that may have two substituents a cycloalkyl group may be attached as a spiro group. Examples of cycloalkyl groups include cyclopropyl, cyclobutyl, cyclopentyl, or cyclohexyl, as well as bridged or caged saturated ring groups such as norbornane or adamantane.

In the term “(cycloalkyl)alkyl”, cycloalkyl and alkyl are as defined above, and the point of attachment is on the alkyl group. This term encompasses, but is not limited to, cyclopropylmethyl, cyclohexylmethyl, and cyclohexylmethyl. Cycloalkyl(C₀-C₂alkyl)” indicates a cycloalkyl group that is directly attached via a single covalent bond cycloalkyl(C₀alkyl) or attached through an alkyl group having from 1 to about 2 carbon atoms.

As used herein “Haloalkyl” indicates both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

“Halo” or “halogen” as used herein refers to fluoro, chloro, bromo, or iodo.

As used herein “hydroxyalkyl” is an alkyl group as defined herein, having the indicated number of carbon atoms, and substituted with at least one hydroxyl substituent (—OH). When indicated, hydroxyalkyl groups, like other groups described herein, may be additionally substituted.

The terms “heterocyclic group” or “heterocyclic ring” indicate a 5-8 membered saturated, partially unsaturated, or aromatic ring containing from 1 to about 4 heteroatoms chosen from N, O, and S, with remaining ring atoms being carbon or a 7-11 membered bicyclic saturated, partially unsaturated, or aromatic heterocylic ring system or a 10 to 15-membered tricyclic ring system, containing at least 1 heteroatom in the multiple ring system chosen from N, O, and S and containing up to about 4 heteroatoms independently chosen from N, O, and S in each ring of the multiple ring system. Unless otherwise indicated, the heterocyclic ring may be attached to its pendant group at any heteroatom or carbon atom that results in a stable structure. When indicated the heterocyclic rings described herein may be substituted on carbon or on a nitrogen atom if the resulting compound is stable. A nitrogen atom in the heterocycle may optionally be quaternized. It is preferred that the total number of heteroatoms in a heterocyclic groups is not more than 4 and that the total number of S and O atoms in a heterocyclic group is not more than 2, more preferably not more than 1. In certain circumstances 5- to 6-membered heterocyclic rings are preferred. Examples of heterocyclic groups include, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyridinyl, pyrimidinyl, furanyl, thienyl, pyrrolyl, pyrazolyl, pyrrolidinyl, morpholinyl, piperazinyl, piperidinyl, and pyrrolidinyl.

As used herein, “heteroaryl” indicates a stable 5- to 7-membered monocyclic aromatic ring which contains from 1 to 3, or preferably from 1 to 2, heteroatoms chosen from N, O, and S, with remaining ring atoms being carbon or a stable bicyclic or tricyclic system containing at least one 5- to 7-membered aromatic ring which contains from 1 to 3, or preferably from 1 to 2, heteroatoms chosen from N, O, and S, with remaining ring atoms being carbon. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Examples of heteroaryl groups include, but are not limited to, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

In the term “heteroarylalkyl,” heteroaryl and alkyl are as defined above, and the point of attachment is on the alkyl group. This term encompasses, but is not limited to, pyridylmethyl, thiophenylmethyl, and pyrrolyl(1-ethyl).

The term “heterocycloalkyl” indicates a saturated monocyclic group containing from 1 to about 3 heteroatoms chosen from N, O, and S, with remaining ring atoms being carbon, or a saturated bicyclic ring system having at least one N, O, or S ring atom with remaining atoms being carbon. Monocyclic heterocycloalkyl groups have from 4 to about 8 ring atoms, and more typically have from 5 to 7 ring atoms. Bicyclic heterocycloalkyl groups typically have from about five to about 12 ring atoms. Preferred heterocycloalkyl groups include 3- to 7-membered monocyclic heterocycloalkyl groups and C₅-C₁₀ bicyclic heterocycloalkyl groups. Examples of heterocycloalkyl groups include morpholinyl, piperazinyl, piperidinyl, and pyrrolidinyl groups.

“Pharmaceutically acceptable forms” of the compounds recited herein are pharmaceutically acceptable salts, hydrates, solvates, crystal forms, polymorphs, chelates, non-covalent complexes, esters, clathrates and prodrugs of such compounds. Pharmaceutically acceptable forms are a preferred pharmaceutically acceptable form. As used herein, a pharmaceutically acceptable salt is an acid or base salt that is generally considered in the art to be suitable for use in contact with the tissues of human beings or animals without excessive toxicity, irritation, allergic response, or other problem or complication.

“Pharmaceutically acceptable salts” includes derivatives of the disclosed compounds wherein the parent compound is modified by making non-toxic acid or base salts thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

The term “prodrugs” includes any compounds that become compounds of Formula 1 when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of Formula 1.

The term “therapeutically effective amount” of a compound of this invention means an amount effective, when administered to a human or non-human patient, to provide a therapeutic benefit such as an amelioration of symptoms, e.g., an amount effective to decrease the symptoms of a viral infection, and preferably an amount sufficient to reduce the symptoms of an HCV infection. In certain circumstances a patient suffering from a viral infection may not present symptoms of being infected. Thus a therapeutically effective amount of a compound is also an amount sufficient to prevent a significant increase or significantly reduce the detectable level of virus or viral antibodies in the patient's blood, serum, or tissues. A significant increase or reduction in the detectable level of virus or viral antibodies is any detectable change that is statistically significant in a standard parametric test of statistical significance such as Student's T-test, where p<0.05.

A “replicon” as used herein includes any genetic element, for example, a plasmid, cosmid, bacmid, phage or virus, that is capable of replication largely under its own control. A replicon may be either RNA or DNA and may be single or double stranded.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule can be described herein according to the normal convention of providing the sequence in the 5′ to 3′ direction.

Viral Inhibitors

The invention provides compounds and salts of Formula 1, also disclosed above,

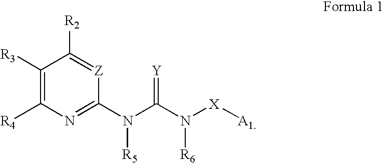

The invention also provides compounds of Formula 1-A

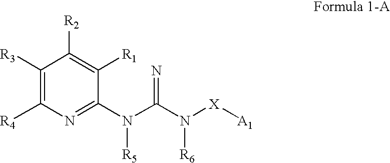

and the pharmaceutically acceptable salts thereof, wherein:

X is absent, —CR′R″—, —(CR′R″)₂—, —CR′R″O—, —O—, or NR.

R is hydrogen, C₁-C₆alkyl, or cyano.

R′ and R″ are independently hydrogen, halogen, C₁-C₂alkyl, or C₁-C₂alkoxy.

R₁, R₂, R₃, and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); where at least one of R₁, R₂, R₃, or R₄ is not hydrogen.

Or, any two of R₁, R₂, R₃ and R₄ which are bound to adjacent carbon atoms are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino, and the remaining two of R₁, R₂, R₃, and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); where at least one of R₁, R₂, R₃, or R₄ is not hydrogen.

R₅ and R₆ are independently hydrogen, C₁-C₄alkyl, C₂-C₄alkenyl, or C₃-C₈cycloalkyl(C₀-C₂alkyl).

A₁ is

i) phenyl, which is substituted with 1 LA₂ substituent and 0 or 1 or more R₇ substituents; or

ii) naphthyl, indanyl, or 9H-fluorenyl, each of which is substituted with 0 or 1 LA₂ substituent, and 0 or 1 or more R₇ substituents; or

iii) heteroaryl, which is substituted with 0 or 1 LA₂ substituent, and 0 or 1 or more R₇ substituents.

R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy).

L is absent, —CR₈R₉—, —(CR₈R₉)₂—, —CR₈R₉O—, —OCR₈R₉—, —O(CR₈R₉)₂—, —NH(C═O)—, —NH(SO₂)—, —O—, or NR₁₀; where R₈ and R₉ are independently hydrogen, halogen, C₁-C₂alkyl, or C₁-C₂alkoxy.

R₁₀ is hydrogen, C₁-C₆alkyl, or cyano.

A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- and di-(C₁-C₆alkyl)amino, mono- and di-(C₁-C₄alkyl)amino(C₁-C₄alkyl), and mono- and di-(C₁-C₄alkyl)amino(C₁-C₄alkoxy).

Also provided herein are compounds and salts if Formula 1-B

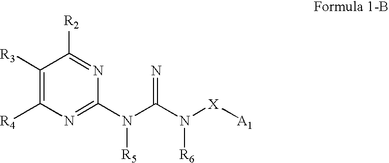

wherein:

X is absent, —CR′R″—, —(CR′R″)₂—, —CR′R″O—, —O—, or NR;

R is hydrogen, C₁-C₆alkyl, or cyano.

R′ and R″ are independently hydrogen, halogen, C₁-C₂alkyl, or C₁-C₂alkoxy.

R₂ is C₁-C₂alkyl, trifluoromethyl, or C₁-C₂alkoxy.

R₃ and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy).

Or, R₃ and R₄ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino.

R₅ and R₆ are independently hydrogen, C₁-C₄alkyl, C₂-C₄alkenyl, or C₃-C₈cycloalkyl(C₀-C₂alkyl).

A₁ is

i) phenyl, which is substituted with 1 LA₂ substituent and 0 or 1 or more R₇ substituents; or

ii) naphthyl, indanyl, or 9H-fluorenyl, each of which is substituted with 0 or 1 LA₂ substituent, and 0 or 1 or more R₇ substituents; or

iii) heteroaryl, which is substituted with 0 or 1 LA₂ substituent, and 0 or 1 or more R₇ substituents.

R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy).

L is absent, —CR₈R₉—, —(CR₈R₉)₂—, —CR₈R₉O—, —OCR₈R₉—, —O(CR₈R₉)₂—, —NH(C═O)—, or —O, where R₈ and R₉ are independently hydrogen, halogen, C₁-C₂alkyl, or C₁-C₂alkoxy.

A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino, with the proviso that when R₂ and R₄ are both methyl and R₃ is hydrogen, and with the proviso that LA₂ is not phenoxy, phenylethoxy, 5-dimethylbenzo[d]thiazolyl, allyloxy, piperidin-1-ylsulfonyl, or pentoxy.

In certain compound and forms thereof described herein X is absent, —CR′R″—, or —(CR′R″)₂—. In other embodiments X is absent, —CH₂—, or —(CH₂)₂—.

Provided herein are compounds and forms thereof wherein Z is N. Also provided herein are compounds and forms thereof in which Z is CR₁.

The Variables R₁, R₂, R₃, and R₄

In certain compounds and forms thereof provided herein the variables R₁-R₄ satisfy the following conditions:

R₂ and R₄ are hydrogen, halogen, hydroxy, cyano, nitro, amino, acetyl, oxo, C₁-C₆alkyl, C₂-C₆alkenyl, C₁-C₆alkoxy, C₁-C₆hydroxyalkyl, C₁-C₆aminoalkyl, C₁-C₂haloalkyl, C₁-C₂haloalkoxy, C₂-C₆alkanoyl, C₁-C₄alkylthio, or mono- or di-(C₁-C₆alkyl)amino; and R₃ are hydrogen or methyl.

R₂ and R₄ are hydrogen, halogen, hydroxy, oxo, C₁-C₆alkyl, C₁-C₆alkoxy, C₁-C₂haloalkyl, C₁-C₂haloalkoxy, or mono- or di-(C₁-C₆alkyl)amino; and R₃ is hydrogen or methyl.

R2 and R4 are hydrogen, C1-C2alkyl, C1-C2alkoxy, or trifluoromethyl; and R3 is hydrogen.

R3 is hydrogen or methyl; and R4 is hydrogen, halogen, hydroxy, cyano, nitro, amino, acetyl, oxo, C1-C6alkyl, C2-C6alkenyl, C1-C6alkoxy, C1-C6hydroxyalkyl, C1-C6aminoalkyl, C1-C2haloalkyl, C1-C2haloalkoxy, C2-C6alkanoyl, C1-C4alkylthio, or mono- or di-(C1-C6alkyl)amino.

R3 is hydrogen or methyl; and R4 is hydrogen, halogen, hydroxy, oxo, C1-C6alkyl, C1-C6alkoxy, C1-C2haloalkyl, C1-C2haloalkoxy, or mono- or di-(C1-C6alkyl)amino.

R3 is hydrogen; and R₄ is hydrogen, hydroxy, C₁-C₂alkyl, C₁-C₂alkoxy, or trifluoromethyl.

R1, R2, R3, and R4 are independently hydrogen, halogen, hydroxy, cyano, nitro, amino, acetyl, oxo, C1-C6alkyl, C2-C6alkenyl, C1-C6alkoxy, C1-C6hydroxyalkyl, C1-C6aminoalkyl, C1-C2haloalkyl, C1-C2haloalkoxy, C2-C6alkanoyl, C1-C4alkylthio, or mono- or di-(C1-C6alkyl)amino.

R1, R2, R3, and R4 are independently hydrogen, halogen, hydroxy, oxo, C1-C6alkyl, C1-C6alkoxy, C1-C2haloalkyl, C1-C2haloalkoxy, or mono- or di-(C1-C6alkyl)amino.

R1, R2, R3, and R4 are independently hydrogen, C1-C2alkyl, C1-C2alkoxy, or trifluoromethyl.

Further provided herein are compounds and pharmaceutically acceptable forms of Formula 2

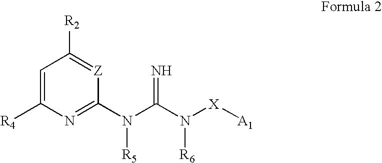

wherein

X is absent, —CH₂—, or —CH₂CH₂—.

Z is CH or N.

R₁, when present is hydrogen.

R₂ and R₄ are independently hydrogen, methyl, and methoxy, wherein at least one of R₂ and R₄ is not hydrogen; and R₅ and R₆ are independently hydrogen or methyl.

Compounds of Formula 1 in which R₃ and R₄ are Joined to Form a Ring

Provided herein are compounds and pharmaceutically acceptable forms thereof in which R₃ and R₄ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino.

In certain embodiments described herein

R₃ and R₄ are joined to form a 6-membered aryl ring or 6-membered heteroaryl ring containing 1 or 2 nitrogen atoms, each of which aryl ring or heteroaryl ring is substituted with 0, or 1 or more substituents chosen from halogen, C₁-C₂alkyl, C₁-C₂alkoxy, trifluoromethyl, or trifluoromethoxy.

In other embodiments described herein

R₃ and R₄ are joined to form a phenyl ring substituted with 0, or 1 or more substituents chosen from halogen, C₁-C₂alkyl, C₁-C₂alkoxy, trifluoromethyl, or trifluoromethoxy.

Also described herein are compounds and pharmaceutically acceptable forms thereof wherein

R₃ and R₄ are joined to form a 6-membered cycloalkyl ring or 6-membered heterocycloalkyl ring containing 1 or 2 N, O, or S heteroatoms, each of which cycloalkyl ring or heterocycloalkyl ring is substituted with 0, or 1 or more substituents chosen from halogen, C₁-C₂alkyl, C₁-C₂alkoxy, trifluoromethyl, or trifluoromethoxy.

In certain embodiments described herein

R₃ and R₄ are joined to form a cyclohexyl ring, substituted with 0, or 1 or more substituents chosen from halogen, C₁-C₂alkyl, C₁-C₂alkoxy, trifluoromethyl, or trifluoromethoxy.

When R₃ and R₄ are joined to form a ring it is preferred that:

R₁ and R₂ are independently hydrogen, halogen, hydroxy, cyano, nitro, amino, acetyl, oxo, C₁-C₆alkyl, C₂-C₆alkenyl, C₁-C₆alkoxy, C₁-C₆hydroxyalkyl, C₁-C₆aminoalkyl, C₁-C₂haloalkyl, C₁-C₂haloalkoxy, C₂-C₆alkanoyl, C₁-C₄alkylthio, or mono- or di-(C₁-C₆alkyl)amino.

Or that R1 is hydrogen, halogen, hydroxy, cyano, nitro, amino, acetyl, oxo, C1-C6alkyl, C2-C6alkenyl, C1-C6alkoxy, C1-C6hydroxyalkyl, C1-C6aminoalkyl, C1-C2haloalkyl, C1-C2haloalkoxy, C2-C6alkanoyl, C1-C4alkylthio, or mono- or di-(C1-C6alkyl)amino.

Or that, R1 is hydrogen, halogen, hydroxy, oxo, C1-C6alkyl, C1-C6alkoxy, C1-C2haloalkyl, C1-C2haloalkoxy, or mono- or di-(C1-C6alkyl)amino, and R2 is methyl or methoxy.

Or that R1 is hydrogen, C1-C2alkyl, C1-C2alkoxy, or trifluoromethyl, and R2 is methyl.

Or, more preferably wherein

R₁ and R₂ are independently hydrogen, halogen, hydroxy, oxo, C₁-C₆alkyl, C₁-C₆alkoxy, C₁-C₂haloalkyl, C₁-C₂haloalkoxy, or mono- or di-(C₁-C₆alkyl)amino.

Or, more preferably still wherein

R1 and R₂ are independently hydrogen, C₁-C₂alkyl, C₁-C₂alkoxy, or trifluoromethyl.

Thus, included herein are compounds and pharmaceutically acceptable forms thereof of Formula 3 and Formula 4 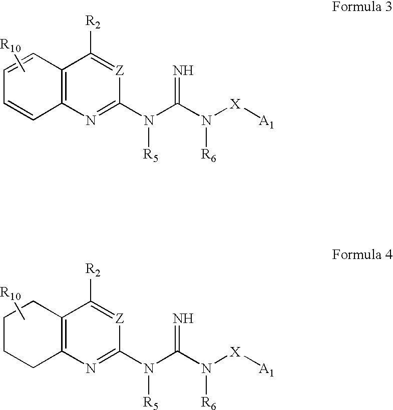
in which:
X is absent, —CH₂—, or —CH₂CH₂—; Z is N or CH; R₂ is methyl or methoxy; R₅ and R₆ are independently hydrogen or methyl; and R₁₀ represents 0 to 3 substitutents independently chosen from halogen, C₁-C₂alkyl, and C₁-C₂alkoxy.
Compounds of Formula 1 in which R₂ and R₃ are Joined to Form a Ring

Provided herein, are compounds and pharmaceutically acceptable forms thereof, in which:

R₂ and R₃ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino.

Thus included herein are compounds and pharmaceutically acceptable forms of Formula 5 and Formula 6 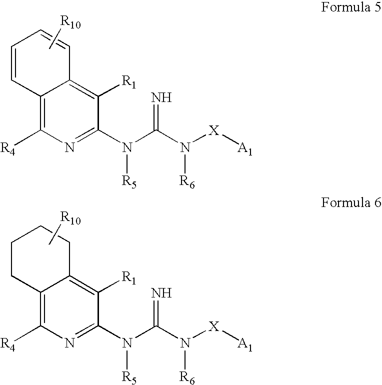
wherein
X is absent, —CH₂—, or —CH₂CH₂—; R₁ and R₄ are independently hydrogen, methyl, or methoxy; R₅ and R₆ are independently hydrogen or methyl; and R₁₀ represents 0 to 3 substitutents independently chosen from halogen, C₁-C₂alkyl, and C₁-C₂alkoxy.
The R₅ and R₆ Variables

Included herein are compounds and pharmaceutically acceptable forms of Formula 1 wherein R₅ and R₆ are independently hydrogen or C₁-C₄alkyl. Preferably R₅ and R₆ are independently hydrogen or methyl, and in certain embodiments R₅ and R₆ are both hydrogen.

The A₁ Variable

Provided herein are compounds and salts of Formula 1 in which meet one or more of the following conditions for the A₁ variable:

A₁ is phenyl, which is substituted with 1 LA₂ substituent and 0 or 1 or more R₇ substituents.

A₁ is naphthyl, indanyl, or 9H-fluoren-2-yl, each of which is substituted with 0 or 1 LA₂ substituents, and 0 or 1 or more R₇ substituents.

A₁ is naphthyl, indanyl, or 9H-fluoren-2-yl, each of which is substituted with 0 LA₂ substituents, and 0 or 1 or more R₇ substituents independently chosen from halogen, hydroxy, amino, C₁-C₂alkyl, C₁-C₂alkoxy, C₁-C₂haloalkyl, and C₁-C₂haloalkoxy.

A₁ is heteroaryl, which is substituted with 0 or 1 LA₂ substituent, and 0 or 1 or more R₇ substituents.

A₁ is pyridyl, pyrimidinyl, benzimidazolyl, quinolinyl, isoquinolinyl, benzo[d]thiazolyl, indolyl, chromanyl, or benzo[d][1,3]dioxolyl, each of which is substituted with 0 LA₂ substituents, and 0 or 1 or more R₇ substituents independently chosen from halogen, hydroxy, amino, oxo, —SH, C₁-C₂alkyl, C₁-C₂alkoxy, C₁-C₂haloalkyl, C₁-C₂haloalkoxy, and phenyl.

Certain embodiments described herein pertain to compounds and salts Formula 7 and Formula 8 and the pharmaceutically forms thereof

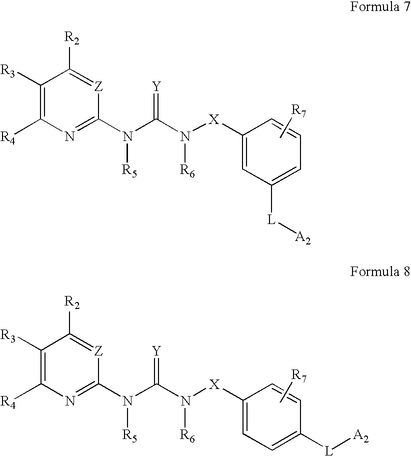

In Formula 7 and Formula 8:

X, Y, Z, and R₂-R₆ carry any of the definitions set forth above for these variables that result in a stable compound.

R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino.

L is absent, —CR₈R₉—, or —O—, where R₈ and R₉ are independently hydrogen, halogen, C₁-C₂alkyl, or C₁-C₂alkoxy.

A₂ is C₃-C₆alkyl or C₃-C₆alkenyl, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- and di-(C₁-C₆alkyl)amino, mono- and di-(C₁-C₄alkyl)amino(C₁-C₄alkyl), and mono- and di-(C₁-C₄alkyl)amino(C₁-C₄alkoxy).

Also included are compounds of Formula 7 and Formula 8 and forms thereof wherein:

R₇ is 0 to 2 substitutents independently chosen from halogen, hydroxy, cyano, C₁-C₂ alkyl, C₁-C₂alkoxy, C₁-C₂haloalkyl, and C₁-C₂haloalkoxy.

L is absent, —CH₂—, or —O—.

A₂ is C₃-C₆alkyl or C₃-C₆alkenyl, each of which is substituted with 0, 1 or more substituents independently chosen from halogen, hydroxy, cyano, amino, oxo, C₁-C₂alkoxy, C₁-C₂haloalkyl, C₁-C₂haloalkoxy, and mono- and di-(C₁-C₄alkyl)amino.

Other embodiments pertain to compounds of Formula 7 and Formula 8 wherein:

R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino.

L is absent, —CR₈R₉—, —(CR₈R₉)₂—, —CR₈R₉O—, —OCR₈R₉—, —O(CR₈R₉)₂—, —NH(C═O)—, or —O—, where R₈ and R₉ are independently hydrogen, halogen, C₁-C₂alkyl, or C₁-C₂alkoxy.

A₂ is phenyl, C₃-C₈cycloalkyl, a monocyclic or bicyclic heteroaryl group having 1, 2, or 3 heteroatoms chosen from N, O, and S, a 5- to 7-membered monocyclic heterocycloalkyl group having 1 N ring atom and 0 or 1 additional heteroatoms chosen from N, O, and S, an 8- to 12-membered bicyclic heterocycloalkyl group having 1 N ring atom and 0 to 2 additional heteroatoms chosen from N, O, and S, or a partially unsaturated monocyclic or bicyclic heterocyclic group having 1 N ring atom and 0 to 2 additional heteroatoms chosen from N, O, and S, each of which A₂ is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- and di-(C₁-C₆alkyl)amino, mono- and di-(C₁-C₄alkyl)amino(C₁-C₄alkyl), and mono- and di-(C₁-C₄alkyl)amino(C₁-C₄alkoxy).

Also included are compounds and forms of Formula 7 and Formula 8 wherein:

R₇ is 0 to 2 substitutents independently chosen from halogen, hydroxy, cyano, C₁-C₂ alkyl, C₁-C₂alkoxy, C₁-C₂haloalkyl, and C₁-C₂haloalkoxy.

L is absent, —CH₂—, —(CH₂)₂—, —CH₂O—, —OCH₂—, —O(CH₂)₂—, —NH(C═O)—, or —O—.

Other embodiments pertain to compounds of Formula 7 and Formula 8 wherein:

R₇ is 0 to 2 substitutents independently chosen from halogen, hydroxy, cyano, C₁-C₂ alkyl, C₁-C₂alkoxy, C₁-C₂haloalkyl, and C₁-C₂haloalkoxy.

L is absent, —CH₂—, —(CH₂)₂—, —CH₂O—, —OCH₂—, —O(CH₂)₂—, —NH(C═O)—, or —O—.

A₂ is phenyl, cyclohexyl, cycloheptyl, piperidinyl, piperazinyl, morpholinyl, thiomorpholinyl, pyrrolidinyl, pyridyl, pyrimidinyl, pyrazinyl, imidazolyl, furanyl, thienyl, thiazolyl, oxazolyl, pyrrolyl, quinolinyl, isoquinolinyl, indanyl, dihydroisoquinonlinyl, or octahydroisoquinolinyl, each of which A₂ is substituted with 0 to 3 substituents independently chosen from halogen, hydroxy, cyano, amino, acetyl, oxo, C₁-C₆alkyl, C₂-C₄alkenyl, C₁-C₄alkoxy, C₁-C₂haloalkyl, C₁-C₂haloalkoxy, C₂-C₄alkanoyl, C₁-C₄alkylthio, and mono- and di-(C₁-C₆alkyl)amino.

Further included herein are compounds of Formula 9 to Formula 12 and the pharmaceutically acceptable forms thereof

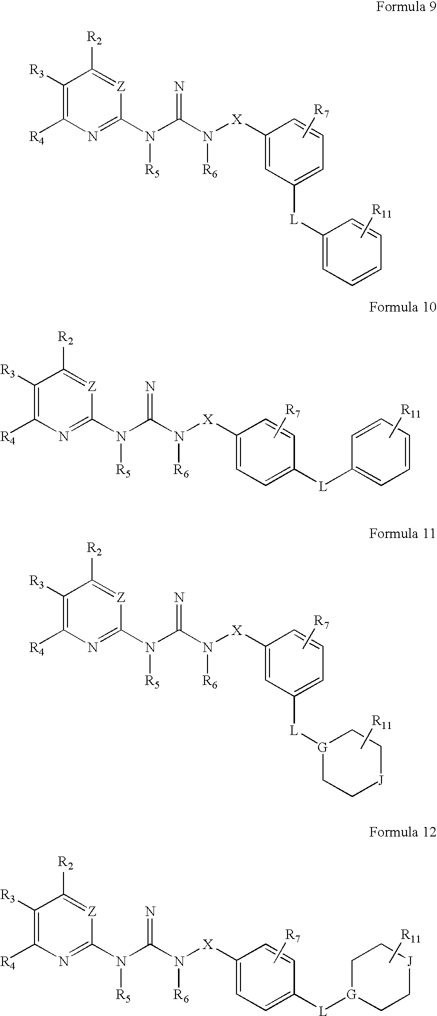

In Formula 9-Formula 12:

X, Z, and R₂-R₆ carry any of the definitions set forth above for these variables that results in a stable compound.

G is CH or N.

J is CH₂, N, O, or S.

X is absent, —CH₂—, or —CH₂CH₂—.

R₅ and R₆ are independently hydrogen or methyl; and

R₁₁ is 0 to 3 substituents independently chosen from halogen, hydroxy, cyano, acetyl, C₁-C₆alkyl, C₂-C₄alkenyl, C₁-C₄alkoxy, C₁-C₂haloalkyl, C₁-C₂haloalkoxy, C₁-C₄alkylthio, and mono- and di-(C₁-C₆alkyl)amino.

Without wishing to be bound to any particular theory, it is believed that the anti-HCV activity of compounds of Formula 1 is due to their inhibit replication of the HCV replicon. Preferred compounds of Formula 1 exhibit an EC50 of about 10 micromolar or less, or more preferably an EC50 of about 1 micromolar or less; or an EC50 of about 500 nanomolar or less in an HCV replicon assay.

Preferred compounds of Formula 1 will have certain pharmacological properties. Such properties include, but are not limited to oral bioavailability, low toxicity, low serum protein binding and desirable in vitro and in vivo half-lives.

The invention includes packaged pharmaceutical formulations. Such packaged formulations include a pharmaceutical composition containing one or more compounds or salts of Formula 1 in a container and instructions for using the composition to treat a patient suffering from Hepatitis C infection (HCV infection).

Pharmaceutical Preparations

Compounds and salts of the invention can be administered as the neat chemical, but are preferably administered as a pharmaceutical composition or formulation. Accordingly, the invention provides pharmaceutical formulations comprising a compound or pharmaceutically acceptable salt of Formula 1, together with one or more pharmaceutically acceptable carriers, excipients, adjuvant, diluent, or other ingredients.

In addition to one or more compounds of the invention, pharmaceutical compositions of the invention may contain a pharmaceutically acceptable carrier, one or more compatible solid or liquid filler diluents or encapsulating substances, which are suitable for administration to a patient. Carriers must be of sufficiently high purity and sufficiently low toxicity to render them suitable for administration to the animal being treated. The carrier can be inert or it can possess pharmaceutical benefits. The amount of carrier employed in conjunction with the compound is sufficient to provide a practical quantity of material for administration per unit dose of the compound.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, and corn oil; polyols such as propylene glycol, glycerine, sorbitol, mannitol, and polyethylene glycol; alginic acid; emulsifiers, such as the TWEENS; bioavailability enhancers, such as lauroyl macroglycerides, including GELUCIRE, wetting agents, such sodium lauryl sulfate; coloring agents; flavoring agents; tableting agents, stabilizers; antioxidants; preservatives; pyrogen-free water; isotonic saline; and phosphate buffer solutions.

Optional active agents may be included in a pharmaceutical composition, which do not substantially interfere with the activity of the compounds of the present invention.

Effective concentrations of one or more of the compounds of the invention including pharmaceutically acceptable salts, esters or other derivatives thereof are mixed with a suitable pharmaceutical carrier, excipients, adjuvant, or vehicle. In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as salts of the compounds or prodrugs of the compounds may also be used in formulating effective pharmaceutical compositions.

Upon mixing or addition of the compound(s) of the invention, the resulting mixture may be a solution, suspension, emulsion or the like. The form of the resulting mixture depends upon a number of factors, including the intended mode of administration and the solubility of the compound in the chosen carrier or vehicle. The effective concentration sufficient for ameliorating the symptoms of the disease, disorder, or condition treated and may be empirically determined.

Compounds of general the invention may be administered orally, topically, parenterally, by inhalation or spray, sublingually, transdermally, via buccal administration, rectally, as an ophthalmic solution, or by other means, in dosage unit formulations.

Dosage formulations suitable for oral use, include, for example, tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations. Oral formulations contain between 0.1 and 99% of a compound of the invention and usually at least about 5% (weight %) of a compound of the present invention. Some embodiments contain from about 25% to about 50% or from 5% to 75% of a compound of invention.

Orally administered compositions also include liquid solutions, emulsions, suspensions, powders, granules, elixirs, tinctures, syrups, and the like. The pharmaceutically acceptable carriers suitable for preparation of such compositions are well known in the art. Oral formulations may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

Orally Administered Liquids formulations

Compounds of the invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Suspensions

For a suspension, typical suspending agents include methylcellulose, sodium carboxymethyl cellulose, AVICEL RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Emulsions

Pharmaceutical compositions of the invention may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

Dispersible Powders

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Injectable and Parenteral Formulations

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid are useful in the preparation of injectables.

Compounds of the invention may be administered parenterally in a sterile medium. Parenteral administration includes subcutaneous injections, intravenous, intramuscular, intrathecal injection or infusion techniques. The compound or compounds of the invention, depending on the vehicle and concentration used, can either be suspended or dissolved in the vehicle. Advantageously, adjuvants such as local anesthetics, preservatives and buffering agents can be dissolved in the vehicle. In many compositions for parenteral administration the carrier comprises at least about 90% by weight of the total composition. Preferred carriers for parenteral administration include propylene glycol, ethyl oleate, pyrrolidone, ethanol, and sesame oil.

Suppositories

Compounds of the invention may also be administered in the form of suppositories for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Topical Formulations

Compounds of the invention may be formulated for local or topical application, such as for topical application to the skin and mucous membranes, such as in the eye, in the form of gels, creams, and lotions and for application to the eye or for intracisternal or intraspinal application. Topical compositions of the present invention may be in any form including, for example, solutions, creams, ointments, gels, lotions, milks, cleansers, moisturizers, sprays, skin patches, and the like.

Such solutions may be formulated as 0.01%-10% isotonic solutions, pH about 5-7, with appropriate salts. Compounds of the invention may also be formulated for transdermal administration as a transdermal patch.

Topical compositions containing the active compound can be admixed with a variety of carrier materials well known in the art, such as, for example, water, alcohols, aloe vera gel, allantoin, glycerine, vitamin A and E oils, mineral oil, propylene glycol, PPG-2 myristyl propionate, and the like.

Other materials suitable for use in topical carriers include, for example, emollients, solvents, humectants, thickeners and powders. Examples of each of these types of materials, which can be used singly or as mixtures of one or more materials, are as follows:

Emollients, such as stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, filmed silica, carboxyvinyl polymer, sodium carboxymethyl cellulose, and ethylene glycol monostearate.

Compounds described herein may also be topically administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Other Formulations

Other compositions useful for attaining systemic delivery of the subject compounds include sublingual, buccal and nasal dosage forms. Such compositions typically comprise one or more of soluble filler substances such as sucrose, sorbitol and mannitol, and binders such as acacia, microcrystalline cellulose, carboxymethyl cellulose, and hydroxypropyl methylcellulose. Glidants, lubricants, sweeteners, colorants, antioxidants and flavoring agents disclosed above may also be included.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Additional Components

The compositions provided herein may also optionally comprise an activity enhancer. The activity enhancer can be chosen from a wide variety of molecules that function in different ways to enhance antimicrobial effects of compounds described herein. Particular classes of activity enhancers include skin penetration enhancers and absorption enhancers.

Pharmaceutical compositions may also contain additional active agents can be chosen from a wide variety of molecules, which can function in different ways to enhance the antimicrobial or therapeutic effects of a compound of the present invention. These optional other active agents, when present, are typically employed in the compositions described herein at a level ranging from about 0.01% to about 15%. Some embodiments contain from about 0.1% to about 10% by weight of the composition. Other embodiments contain from about 0.5% to about 5% by weight of the composition.

Packaged Formulations

Packaged pharmaceutical formulations are included herein. Such packaged formulations include a pharmaceutical composition containing one or more compounds or salts of the invention in a container and instructions for using the composition to treat an animal (typically a human patient) suffering from a microorganism infection or disorder or prevent a microorganism infection in a patient.

The invention includes providing prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the compounds of the invention can be administered alone, as mixtures, or in combination with other active agents.

Methods of Treatment

The invention includes methods of treating viral infections, particularly HCV infections, by administering an effective amount of one or more compounds of Formula 1 to patient suffering from a viral infection. An effective amount of a compound of Formula 1 may be an amount sufficient to reduce the symptoms of viral infection. Alternatively an effective amount of a compound of Formula 1 may be an amount sufficient to significantly reduce the amount of virus or viral antibodies detectable in a patient's tissues or bodily fluids.

Methods of treatment include administering an amount of a compound of Formula 1 sufficient to reduce or relieve the jaundice, fatigue, dark urine, abdominal pain, loss of appetite, and nausea associated with HCV infection.

Compounds of Formula 1 are thought to ameliorate the HCV disease process by virtue of their inhibition of the replication of the Hepatitis C virus. The compounds provided herein may be virucidal, in that they actually kill the active virus, in addition to independently inhibiting viral replication. The provided compounds may also function through mechanisms that involve a combination of virucidal activity and inhibition of replication.

Methods of treatment encompassed by the invention include administering a compound of Formula 1 as the sole active and administering a compound of Formula 1 together with one or more other active agents, such another antiviral agent, particularly an anti-viral agent effective against HCV infection. The invention includes administering one or more compounds of Formula 1 together with Peg-interferon, Peg-interferon alpha 2b, Ribavarin, natural interferon, Albuferon, interferon beta-1a, IL-10, interferon gamma-1b, AMANTADINE, or ZADAXIM.

Methods of treatment also include inhibiting HCV replication in vivo, in a patient infected with HCV, by administering a sufficient concentration of a compound of Formula 1 to inhibit HCV replicon replication in vitro. By “sufficient concentration” of a compound administered to the patient is meant the concentration of the compound available in the patient's system to combat the infection. Such a concentration by be ascertained experimentally, for example by assaying blood concentration of the compound, or theoretically, by calculating bioavailability.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### EXAMPLES

### Synthesis of Compounds

An illustration of the preparation of compounds of the present invention is given in below in Example 1. Those having skill in the art will recognize that the starting materials may be varied and additional steps employed to produce compound encompassed by the present invention.

### Example 1

### Synthesis of 1-(6-METHYLPYRIDIN-2-YL)-3-(4-PHENOXYPHENYL)GUANIDINE

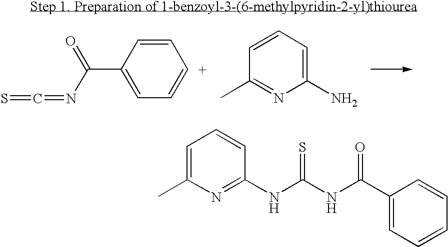

Benzoyl isothiocyanate (0.33 g, 2.0 mmol) is dissolved in 5 mL anhydrous dichloromethane. The solution is added slowly to a solution of 6-methylpicoline (0.23 g, 2.0 mmol) in 2 mL dichloromethane. The resulting mixture is stirred at room temperature for 1 hour. The solvent is evaporated under reduced pressure. The residue is recrystallized from isopropanol to give the product as off-white short needles LCMS, retention time: 1.85 min., M+H⁺: 272

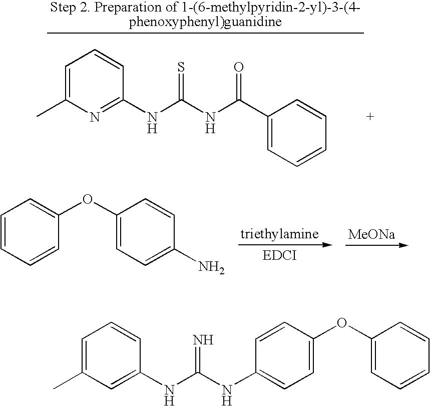

N-benzoyl-N′-[2-(6-methyl)pyridinyl]thiourea (27 mg, 0.1 mmol), 4-phenoxyaniline (18 mg, 0.1 mmol), EDCI (22.5 mg, 0.12 mmol), and triethylamine (20 μL, 0.14 mmol) are stirred in 1 mL dichloromethane at room temperature for 24 hours. Sodium methoxide (0.15 mL 25% w/w, 0.75 mmol) and 0.1 mL methanol is added to the above reaction mixture. The mixture is stirred at room temperature for additional 48 hours. Hydrochloric acid in methanol (6 N, 0.125 mL, 0.75 mmol) is added. After filtration, the resulting reaction mixture is purified by preparative LCMS to give the product as clear oil (LCMS, retention time: 1.59 min, M+H⁺: 319. H¹-NMR (DMSO, σ ppm): 11.20 (br s, 1H), 11.05 (br s, 1H), 9.20-8.60 (br s, 2H), 7.72 (t, 1H), 7.40-7.25 (m, 4H), 7.15-6.84 (m, 7H), 2.35 (s, 3H).)

### Example 2

### Preparation of Additional Compounds of Formula 1

The compounds shown in Table 1 are prepared by method given in example 1.

Retention time (RT) is measured in in a gradient of 30-100% B in 3.00 min; buffer A was 0.1% trifluoroacetic acid in water and buffer B was 0.1% trifluoroacetic acid in acetonitrile. An analytical YMC Pack Pro C18 column was used with a flow rate of 2.5 mL/min. All HPLC/MS analytical runs were run at a wavelength of 220 nm using a Gilson 151 UV/VIS detector followed by a ThermoFinnigan Surveyor MSQ.

| | | | | RT |
|---|---|---|---|---|
| | STRUCTURE | NAME | M + 1 | (min) |
| 1 | 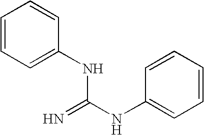 | 1,3- diphenylguanidine | | |
| 2 | 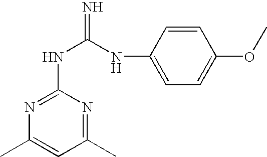 | 1-(4,6- dimethylpyrimidin-2- yl)-3-(4- methoxyphenyl) guanidine | 272 | 1.31 |
| 3 | 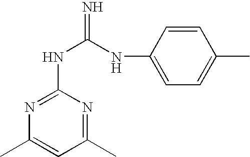 | 1-(4,6- dimethylpyrimidin-2- yl)-3-p- tolylguanidine | | |
| 4 | 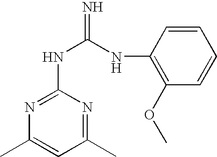 | 1-(4,6- dimethylpyrimidin-2- yl)-3-(2- methoxyphenyl) guanidine | | |
| 5 | 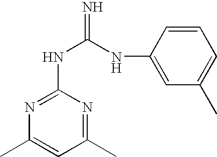 | 1-(4,6- dimethylpyrimidin-2- yl)-3-m- tolylguanidine | | |
| 6 | 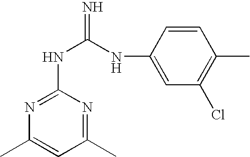 | 1-(3-chloro-4-methyl phenyl)-3-(4,6- dimethyl pyrimidin- 2-yl)guanidine | | |
| 7 | 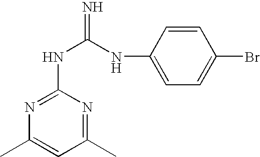 | 1-(4-bromophenyl)- 3-(4,6- dimethylpyrimidin-2- yl)guanidine | | |
| 8 | 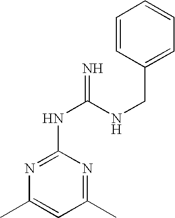 | 1-benzyl-3-(4,6- dimethylpyrimidin-2- yl)guanidine | | |
| 9 | 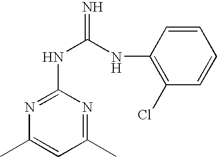 | 1-(2-chlorophenyl)- 3-(4,6- dimethylpyrimidin-2- yl)guanidine | | |
| 10 | 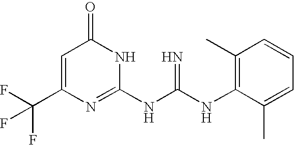 | 1-(2,6- dimethylphenyl)-3- (6-oxo-4- (trifluoromethyl)-1,6- dihydropyrimidin-2- yl)guanidine | | |
| 11 | 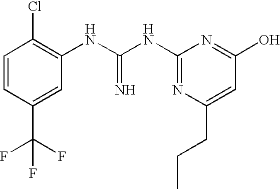 | 1-(2-chloro-5- (trifluoromethyl)phenyl)- 3-(4-hydroxy-6- propylpyrimidin-2- yl)guanidine | | |
| 12 | 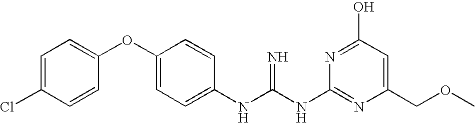 | 1-(4-(4- chlorophenoxy)phen yl)-3-(4-hydroxy-6- (methoxymethyl) pyrimidin-2-yl)guanidine | | |
| 13 | 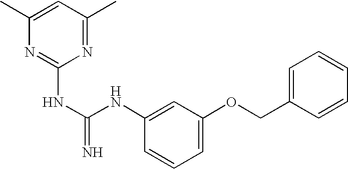 | 1-(3- (benzyloxy)phenyl)- 3-(4,6- dimethylpyrimidin-2- yl)guanidine | | |
| 14 | 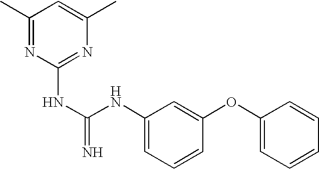 | 1-(4,6- dimethylpyrimidin-2- yl)-3-(3- phenoxyphenyl) guanidine | 333 | 1.31 |
| 15 | 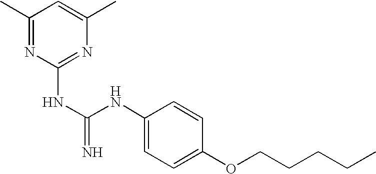 | 1-(4,6- dimethylpyrimidin-2- yl)-3-(4-(pentyloxy) phenyl)guanidine | 327 | 1.51 |
| 16 | 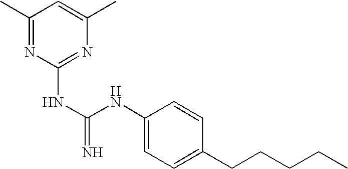 | 1-(4,6- dimethylpyrimidin-2- yl)-3-(4- pentyiphenyl) guanidine | | |
| 17 | 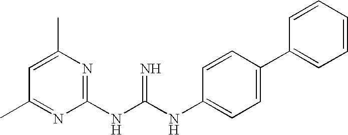 | 1-(4,6- dimethylpyrimidin-2- yl)-3-(4-phenyl- phenyl)guanidine | 318 | 1.17 |
| 18 | 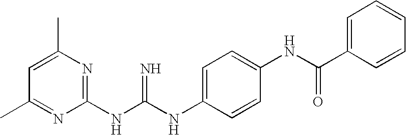 | N-(4-(3-(4,6- dimethylpyrimidin-2- yl)guanidino)phenyl) benzamide | | |
| 19 | 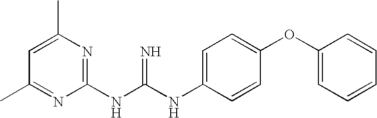 | 1-(4,6- dimethylpyrimidin-2- yl)-3-(4- phenoxyphenyl) guanidine | | |
| 20 | 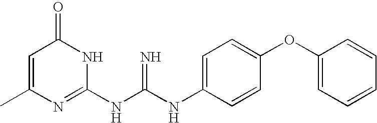 | 1-(4-methyl-6-oxo- 1,6- dihydropyrimidin-2- yl)-3-(4- phenoxyphenyl) guanidine | | |
| 21 | 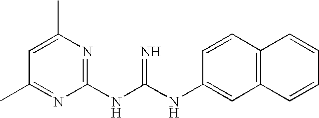 | 1-(4,6- dimethylpyrimidin-2- yl)-3-(naphthalen-2- yl)guanidine | | |
| 22 | 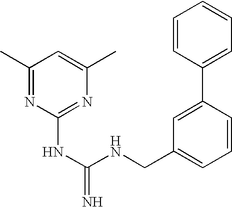 | 1-(3-phenyl-benzyl)- 3-(4,6- dimethylpyrimidin-2- yl)guanidine | 332 | 1.36 |
| 23 | 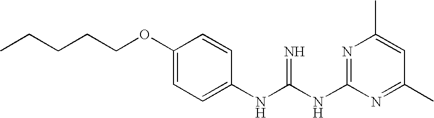 | 1-(4,6- dimethylpyrimidin-2- yl)-3-(4-(pentyloxy) phenyl)guanidine | | |
| 24 | 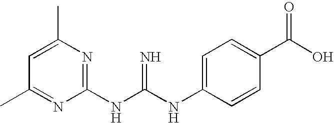 | 4-(3-(4,6-dimethyl pyrimidin-2- yl)guanidino) benzoic acid | | |
| 25 | 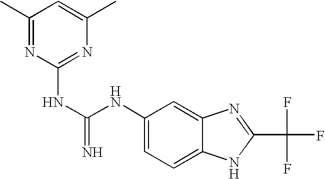 | 1-(4,6- dimethylpyrimidin-2- yl)-3-(2- (trifluoromethyl)- 1H- benzo[d]imidazol-5- yl)guanidine | 348 | 1.48 |
| 26 | 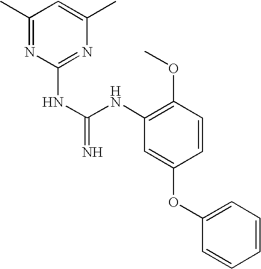 | 1-(4,6- dimethylpyrimidin-2- yl)-3-(2-methoxy-5- phenoxyphenyl) guanidine | 364 | 1.73 |
| 27 | 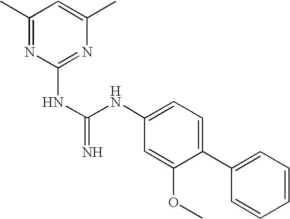 | 1-(4,6- dimethylpyrimidin-2- yl)-3-(3-methoxy-4- phenyl- phenyl)guanidine | 348 | 1.73 |
| 28 | 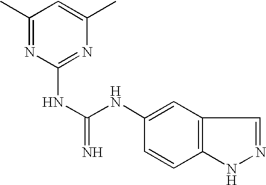 | 1-(4,6- dimethylpyrimidin-2- yl)-3-(1H-indazol-5- yl)guanidine | 282 | 1.4 |
| 29 | 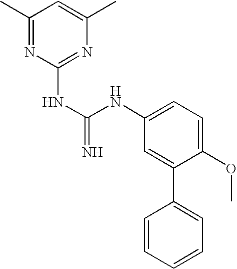 | 1-(4,6- dimethylpyrimidin-2- yl)-3-(3-phenyl-4- methoxyphenyl) guanidine | 348 | 1.72 |
| 30 | 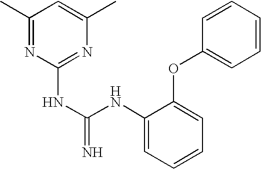 | 1-(4,6- dimethylpyrimidin-2- yl)-3-(2- phenoxyphenyl) guanidine | 334 | 1.67 |
| 31 | 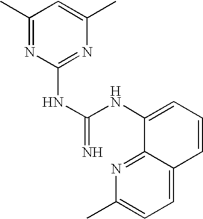 | 1-(4,6- dimethylpyrimidin-2- yl)-3-(2- methylquinolin-8- yl)guanidine | 307 | 1.44 |
| 32 | 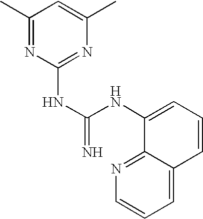 | 1-(4,6- dimethylpyrimidin-2- yl)-3-(quinolin-8- yl)guanidine | 293 | 1.45 |
| 33 | 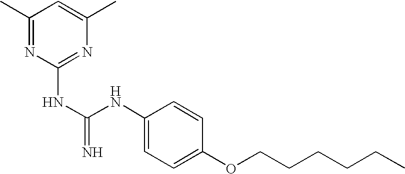 | 1-(4,6- dimethylpyrimidin-2- yl)-3-(4-(hexyloxy) phenyl)guanidine | 342 | 1.87 |
| 34 | 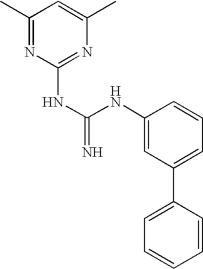 | 1-(4,6- dimethylpyrimidin-2- yl)-3-(3-phenyl- phenyl)guanidine | 318 | 1.7 |
| 35 | 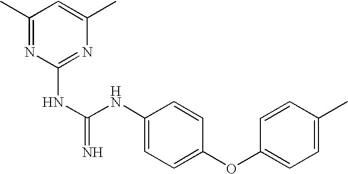 | 1-(4,6- dimethylpyrimidin-2- yl)-3-(4-(p-tolyloxy) phenyl)guanidine | 348 | 1.78 |
| 36 | 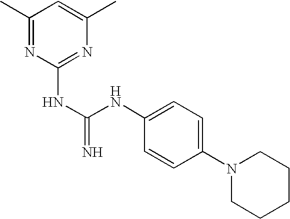 | 1-(4,6- dimethylpyrimidin-2- yl)-3-(4-(piperidin-1- yl)phenyl)guanidine | 325 | 1.28 |
| 37 | 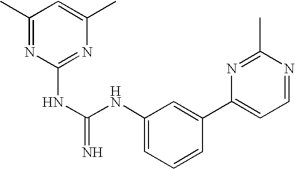 | 1-(4,6- dimethylpyrimidin-2- yl)-3-(3-(2- methylpyrimidlin-4- yl)phenyl)guanidine | 334 | 1.43 |
| 38 | 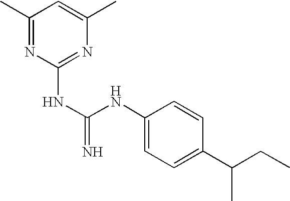 | 1-(4-sec- butylphenyl)-3-(4,6- dimethylpyrimidin-2- yl)guanidine | 298 | 1.75 |
| 39 | 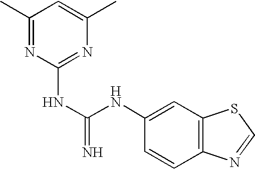 | 1-(benzo[d]thiazol-6- yl)-3-(4,6- dimethylpyrimidin-2- yl)guanidine | 299 | 1.43 |
| 40 | 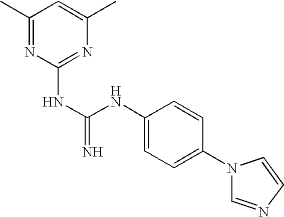 | 1-(4-(1H-imidazol-1- yl)phenyl)-3-(4,6- dimethylpyrimidin-2- yl)guanidine | 308 | 1.2 |
| 41 | 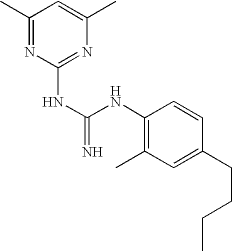 | 1-(4-butyl-2- methylphenyl)-3- (4,6- dimethylpyrimidin-2- yl)guanidine | 312 | 1.8 |
| 42 | 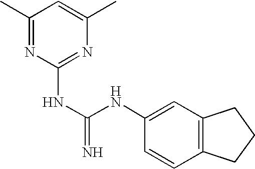 | 1-(2,3-dihydro-1H- inden-5-yl)-3-(4,6- dimethylpyrimidin-2- yl)guanidine | 282 | 1.65 |
| 43 | 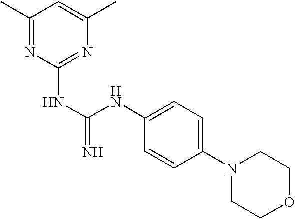 | 1-(4,6- dimethylpyrimidin-2- yl)-3-(4-morpholino- phenyl)guanidine | 327 | 1.46 |
| 44 | 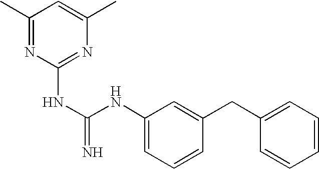 | 1-(3-benzylphenyl)- 3-(4,6- dimethylpyrimidin-2- yl)guanidine | 332 | 1.73 |
| 45 | 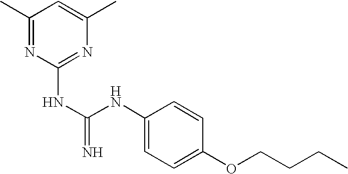 | 1-(4-butoxyphenyl)- 3-(4,6- dimethylpyrimidin-2- yl)guanidine | 314 | 1.76 |
| 46 | 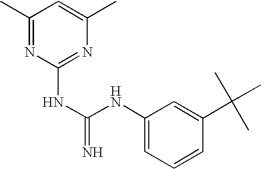 | 1-(3-tert- butylphenyl)-3-(4,6- dimethylpyrimidin-2- yl)guanidine | 298 | 1.73 |
| 47 | 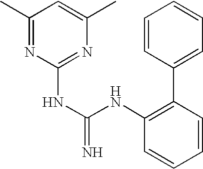 | 1-(4,6- dimethylpyrimidin-2- yl)-3-(2-phenyl- phenyl)guanidine | 318 | 1.67 |
| 48 | 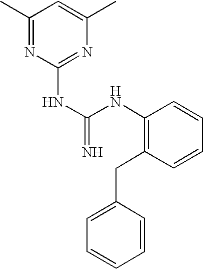 | 1-(2-benzylphenyl)- 3-(4,6- dimethylpyrimidin-2- yl)guanidine | 332 | 1.7 |
| 49 | 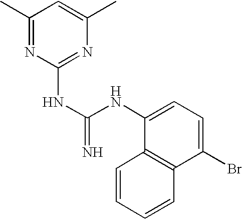 | 1-(4- bromonaphthalen-1- yl)-3-(4,6-dimethyl pyrimidin-2- yl)guanidine | 371 | 1.71 |
| 50 | 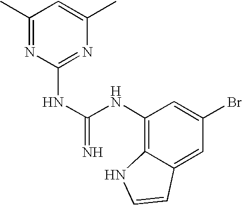 | 1-(5-bromo-1H- indol-7-yl)-3-(4,6- dimethylpyrimidin-2- yl)guanidine | 360 | 1.69 |
| 51 | 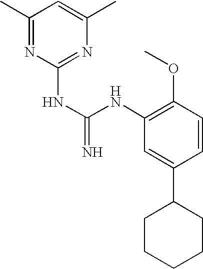 | 1-(5-cyclohexyl-2- methoxyphenyl)-3- (4,6- dimethylpyrimidin-2- yl)guanidine | 354 | 1.83 |
| 52 | 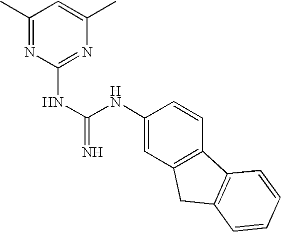 | 1-(4,6- dimethylpyrimidin-2- yl)-3-(9H-fluoren-2- yl)guanidine | 330 | 1.72 |
| 53 | 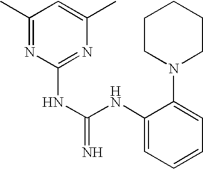 | 1-(4,6- dimethylpyrimidin-2- yl)-3-(2-(piperidin-1- yl)phenyl)guanidine | 325 | 1.67 |
| 54 | 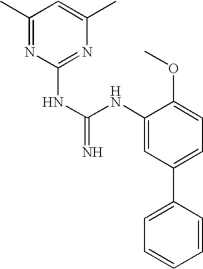 | 1-(4,6- dimethylpyrimidin-2- yl)-3-(2-methoxy-5- phenyl-phenyl) guanidine | 348 | 1.72 |
| 55 | 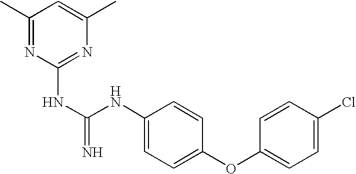 | 1-(4-(4- chlorophenoxy) phenyl)-3-(4,6- dimethyl pyrimidin- 2-yl)guanidine | 368 | 1.79 |
| 56 | 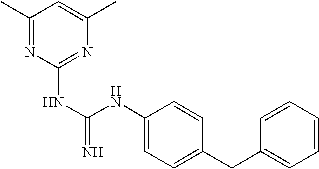 | 1-(4-benzylphenyl)- 3-(4,6- dimethylpyrimidin-2- yl)guanidine | 332 | 1.74 |
| 57 | 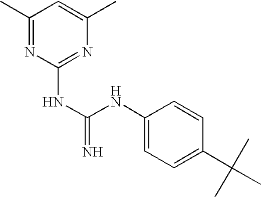 | 1-(4-tert- butylphenyl)-3-(4,6- dimethylpyrimidin-2- yl)guanidine | 298 | 1.73 |
| 58 | 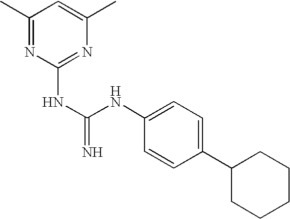 | 1-(4- cyclohexylphenyl)-3- (4,6- dimethylpyrimidin-2- yl)guanidine | 324 | 1.68 |
| 59 | 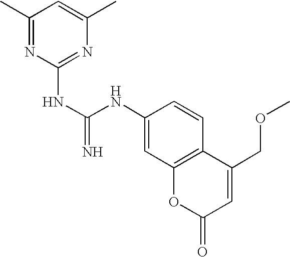 | 1-(4,6- dimethylpyrimidin-2- yl)-3-(4- (methoxymethyl)-2- oxo-2H-chromen-7- yl)guanidine | 354 | 1.31 |
| 60 | 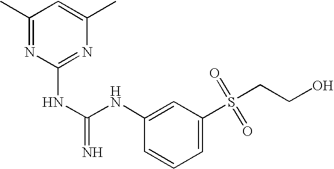 | 1-(4,6- dimethylpyrimidin-2- yl)-3-(3-(2- hydroxyethylsulfonyl) phenyl)guanidine | 350 | 1.17 |
| 61 | 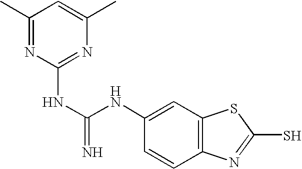 | 1-(4,6- dimethylpyrimidin-2- yl)3-(2- mercaptobenzo[d]thiazol-6-yl)guanidine | 331 | 1.32 |
| 62 | 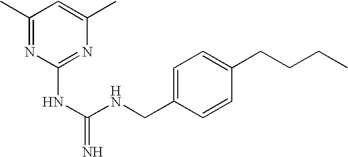 | 1-(4-butylbenzyl)-3- (4,6- dimethylpyrimidin-2- yl)guanidine | 312 | 1.65 |
| 63 | 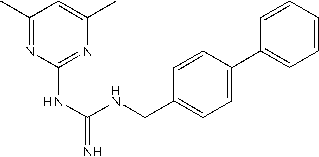 | 1-(4-phenyl-benzyl)- 3-(4,6- dimethylpyrimidin-2- yl)guanidine | 332 | 1.57 |
| 64 | 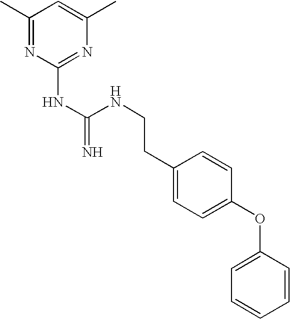 | 1-(4- phenoxyphenethyl)- 3-(4,6- dimethylpyrimidin-2- yl)guanidine | 362 | 1.6 |
| 65 | 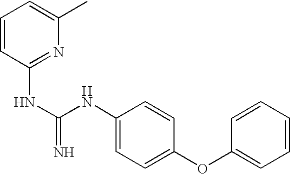 | 1-(6-methylpyridin- 2-yl)-3-(4- phenoxyphenyl) guanidine | 319 | 1.35 |
| 66 | 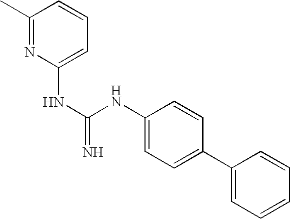 | 1-(6-methylpyridin- 2-yl)-3-(4-phenyl- phenyl)guanidine | 303 | 1.34 |
| 67 | 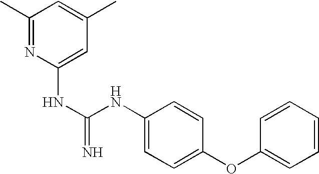 | 1-(4,6- dimethylpyridin-2- yl)-3-(4- phenoxyphenyl) guanidine | 333 | 1.45 |
| 68 | 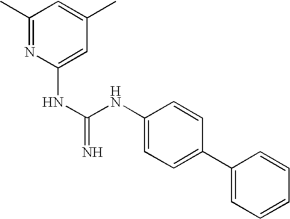 | 1-(4,6- dimethylpyridin-2- yl)-3-(4-phenyl- phenyl)guanidine | 317 | 1.46 |
| 69 | 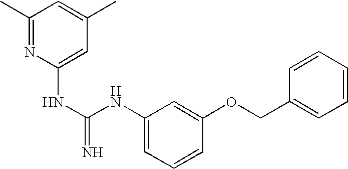 | 1-(3- (benzyloxy)phenyl) 3-(4,6- dimethylpyridin-2- yl)guanidine | 347 | 1.49 |
| 70 | 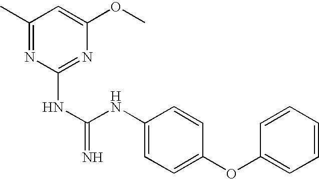 | 1-(4-methoxy-6- methylpyrimidin-2- yl)-3-(4- phenoxyphenyl) guanidine | 350 | 1.27 |
| 71 | 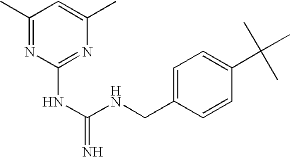 | 1-(4-tert- butylbenzyl)-3-(4,6- dimethylpyrimidin-2- yl)guanidine | 312 | 1.66 |
| 72 | 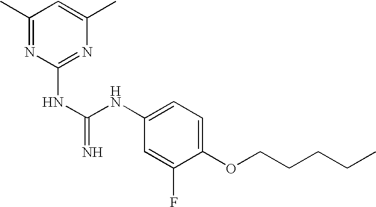 | 1-(4,6- dimethylpyrimidin-2- yl)-3-(3-fluoro-4- (pentyloxy)phenyl) guanidine | 346 | 1.7 |
| 73 | 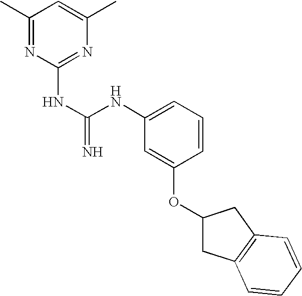 | 1-(3-(indan-2- yloxy)phenyl)-3- (4,6- dimethylpyrimidin-2- yl)guanidine | 374 | 1.69 |
| 74 | 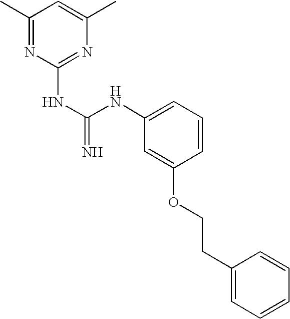 | 1-(4,6- dimethylpyrimidin-2- yl)-3-(3- phenethoxyphenyl) guanidine | 362 | 1.68 |
| 75 | 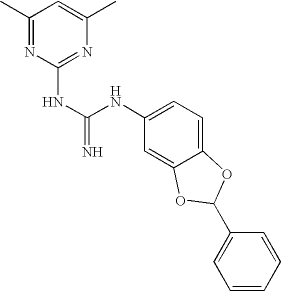 | 1-(4,6- dimethylpyrimidin-2- yl)-3-(2- phenylbenzo[d][1,3]dioxol-5-yl)guanidine | 362 | 1.6 |
| 76 | 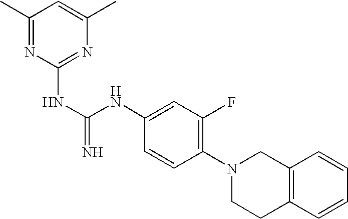 | 1-(4-(3,4- dihydroisoquinolin- 2(1H)-yl)-3- fluorophenyl)-3-(4,6- dimethylpyrimidin-2- yl)guanidine | 391 | 1.68 |
| 77 | 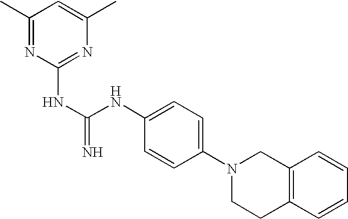 | 1-(4-(3,4- dihydroisoquinolin- 2(1H)-yl)phenyl)-3- (4,6- dimethylpyrimidin-2- yl)guanidine | 373 | 1.62 |
| 78 | 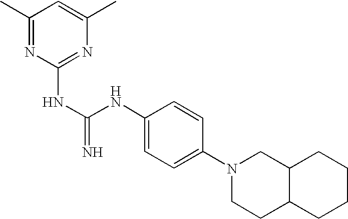 | 1-(4,6- dimethylpyrimidin-2- yl)-3-(4- (octahydroisoquinolin- 2(1H)- yl)phenyl)guanidine | 379 | 1.43 |
| 79 | 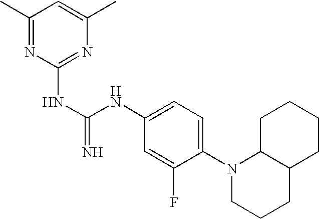 | 1-(4,6- dimethylpyrimidin-2- yl)-3-(3-fluoro-4- (octahydroquinolin- 1(2H)- yl)phenyl)guanidine | 397 | 1.83 |
| 80 | 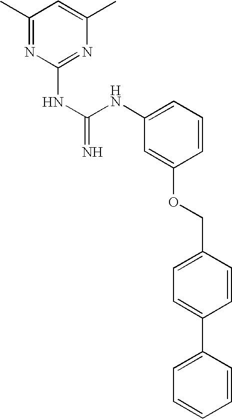 | 1-(3-(4-phenyl- benzyloxy)phenyl) 3-(4,6- dimethylpyrimidin-2- yl)guanidine | 424 | 1.84 |
| 81 | 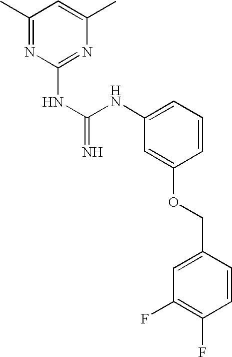 | 1-(3-(3,4- difluorobenzyloxy) phenyl)-3-(4,6- dimethylpyrimidin-2- yl)guanidine | 384 | 1.64 |
| 82 | 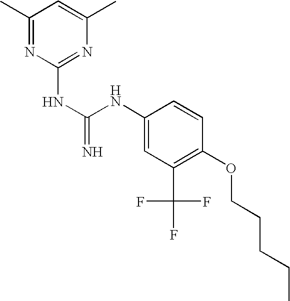 | 1-(4,6- dimethylpyrimidin-2- yl)-3-(4-(pentyloxy)- 3-(trifluoromethyl) phenyl)guanidine | 396 | 1.83 |
| 83 | 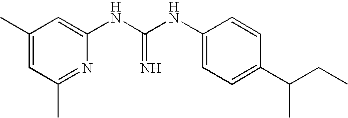 | 1-(4-sec- butylphenyl)-3-(4,6- dimethylpyridin-2- yl)guanidine | 297 | 1.54 |
| 84 | 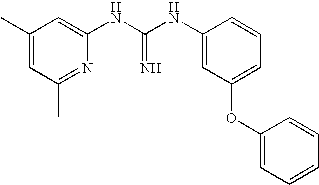 | 1-(4,6- dimethylpyridin-2- yl)-3-(3- phenoxyphenyl) guanidine | 333 | 1.44 |
| 85 | 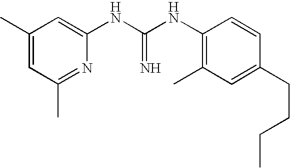 | 1-(4-butyl-2- methylphenyl)-3- (4,6-dimethylpyridin- 2-yl)guanidine | 311 | 1.63 |
| 86 | 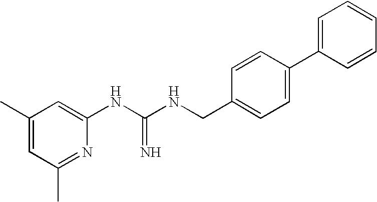 | 1-(4-phenyl-benzyl)- 3-(4,6- dimethylpyridin-2- yl)guanidine | 331 | 1.46 |
| 87 | 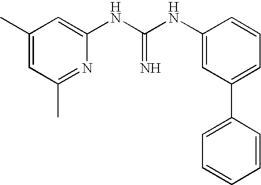 | 1-(4,6- dimethylpyridin-2- yl)-3-(3-phenyl- phenyl)guanidine | 317 | 1.44 |
| 88 | 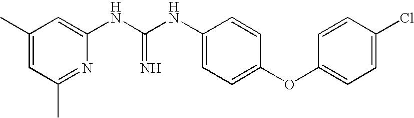 | 1-(4-(4- chlorophenoxy)phenyl)- 3-(4,6- dimethylpyridin-2- yl)guanidine | 367 | 1.59 |
| 89 | 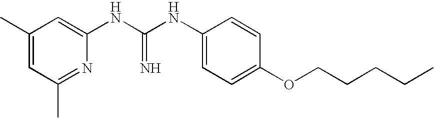 | 1-(4,6- dimethylpyridin-2- yl)-3-(4- (pentyloxy)phenyl) guanidine | 327 | 1.63 |
| 90 | 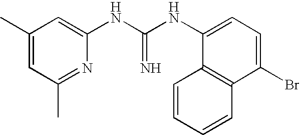 | 1-(4- bromonaphthalen-1- yl)-3-(4,6- dimethylpyridin-2- yl)guanidine | 370 | 1.47 |
| 91 | 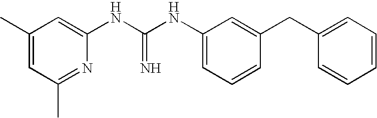 | 1-(3-benzylphenyl)- 3-(4,6- dimethylpyridin-2- yl)guanidine | 331 | 1.49 |
| 92 | 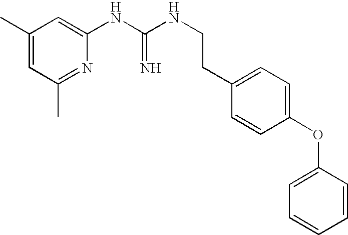 | 1-(4- phenoxyphenethyl)- 3-(4,6- dimethylpyridin-2- yl)guanidine | 361 | 1.51 |
| 93 | 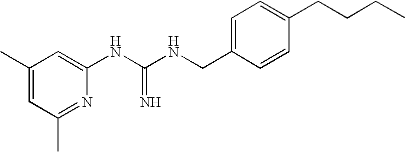 | 1-(4-butylbenzyl)-3- (4,6-dimethylpyridin- 2-yl)guanidine | 311 | 1.61 |
| 94 | 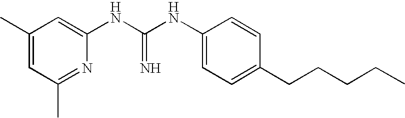 | 1-(4,6- dimethylpyridin-2- yl)-3-(4- pentylphenyl) guanidine | 311 | 1.69 |
| 95 | 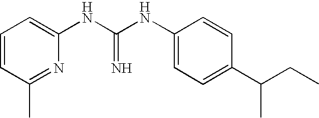 | 1-(4-sec- butylphenyl)-3-(6- methylpyridin-2- yl)guanidine | 283 | 1.42 |
| 96 | 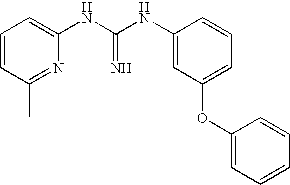 | 1-(6-methylpyridin- 2-yl)-3-(3- phenoxyphenyl) guanidine | 319 | 1.33 |
| 97 | 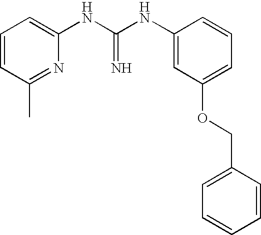 | 1-(3- (benzyloxy)phenyl) 3-(6-methylpyridin- 2-yl)guanidine | 333 | 1.37 |
| 98 | 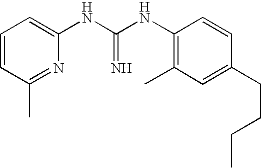 | 1-(4-butyl-2- methylphenyl)-3-(6- methylpyridin-2- yl)guanidine | 297 | 1.52 |
| 99 | 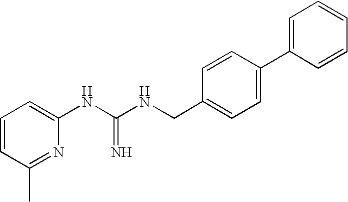 | 1-(4-phenylbenzyl)- 3-(6-methylpyridin- 2-yl)guanidine | 317 | 1.36 |
| 100 | 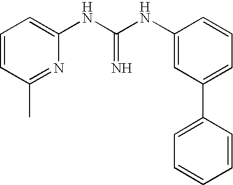 | 1-(6-methylpyridin- 2-yl)-3-(3-phenyl- phenyl)guanidine | 303 | 1.32 |
| 101 | 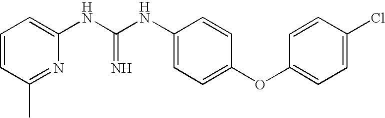 | 1-(4-(4- chlorophenoxy)phenyl)- 3-(6- methylpyridin-2- yl)guanidine | 353 | 1.48 |
| 102 | 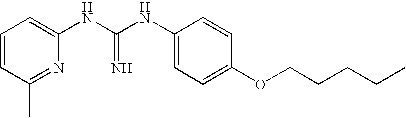 | 1-(6-methylpyridin 2-yl)-3-(4- (pentyloxy)phenyl) guanidine | 313 | 1.5 |
| 103 | 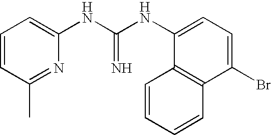 | 1-(4- bromonaphthalen-1- yl)-3-(6- methylpyridin-2- yl)guanidine | 356 | 1.35 |
| 104 | 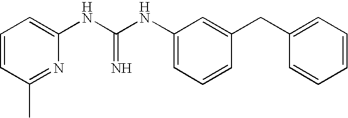 | 1-(3-benzylphenyl) 3-(6-methylpyridin- 2-yl)guanidine | 317 | 1.39 |
| 105 | 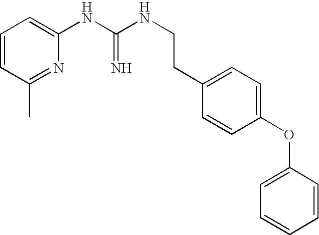 | 1-(4- phenoxyphenethyl)- 3-(6-methylpyridin- 2-yl)guanidine | 356 | 1.35 |
| 106 | 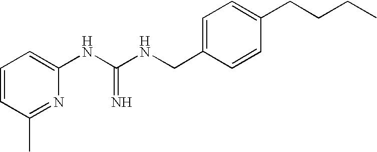 | 1-(4-butylbenzyl)-3- (6-methylpyridin-2- yl)guanidine | 297 | 1.49 |
| 107 | 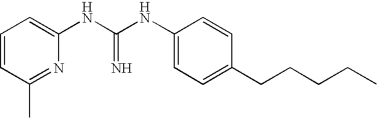 | 1-(6-methylpyridin- 2-yl)-3-(4- pentylphenyl) guanidine | 297 | 1.58 |
| 108 | 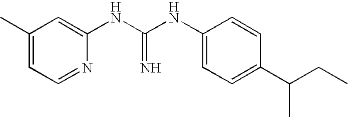 | 1-(4-sec- butylphenyl)-3-(4- methylpyridin-2- yl)guanidine | 283 | 1.4 |
| 109 | 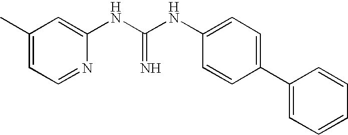 | 1-(4-ethylphenyl)-3- (4-methylpyridin-2- yl)guanidine | 303 | 1.33 |
| 110 | 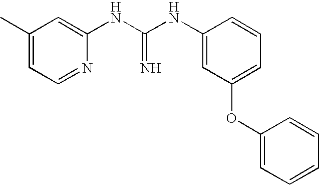 | 1-(4-methylpyridin- 2-yl)-3-(3- phenoxyphenyl) guanidine | 319 | 1.33 |
| 111 | 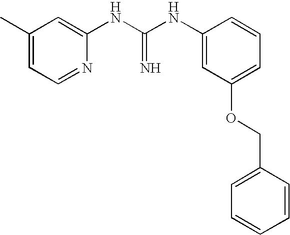 | 1-(3- (benzyloxy)phenyl) 3-(4-methylpyridin- 2-yl)guanidine | 333 | 1.37 |
| 112 | 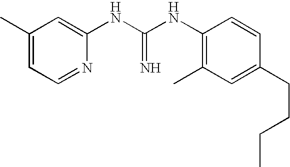 | 1-(4-butyl-2- methylphenyl)-3-(4- methylpyridin-2- yl)guanidine | 297 | 1.52 |
| 113 | 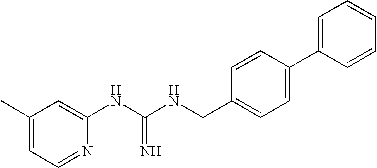 | 1-(4-phenyl-benzyl)- 3-(4-methylpyridin- 2-yl)guanidine | 317 | 1.34 |
| 114 | 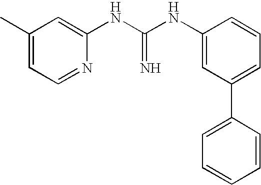 | 1-(3-phenyl-phenyl)- 3-(4-methylpyridin- 2-yl)guanidine | 303 | 1.32 |
| 115 | 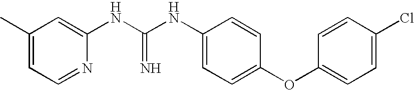 | 1-(4-(4- chlorophenoxy)phenyl)- 3-(4- methylpyridin-2- yl)guanidine | 353 | 1.49 |
| 116 | 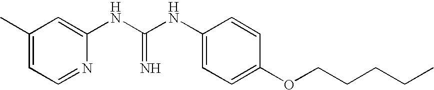 | 1-(4-methylpyridin- 2-yl)-3-(4- (pentyloxy)phenyl) guanidine | 313 | 1.5 |
| 117 | 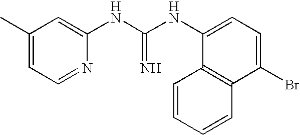 | 1-(4- bromonaphthalen-1- yl)-3-(4- methylpyridin-2- yl)guanidine | 356 | 1.35 |
| 118 | 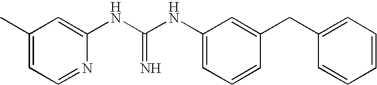 | 1-(3-benzylphenyl)- 3-(4-methylpyridin- 2-yl)guanidine | 317 | 1.37 |
| 119 | 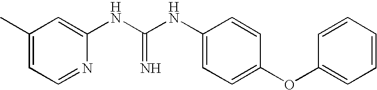 | 1-(4-methylpyridin- 2-yl)-3-(4- phenoxyphenyl) guanidine | 319 | 1.34 |
| 120 | 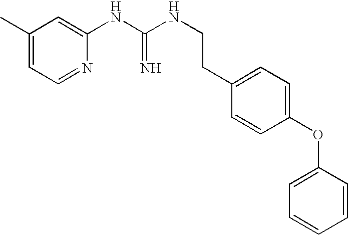 | 1-(4- phenoxyphenethyl)- 3-(4-methylpyridin- 2-yl)guanidine | 347 | 1.41 |
| 121 | 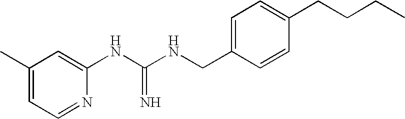 | 1-(4-butylbenzyl)-3- (4-methylpyridin-2- yl)guanidine | 297 | 1.46 |
| 122 | 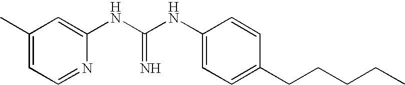 | 1-(4-methylpyridin- 2-yl)-3-(4- pentylphenyl) guanidine | 297 | 1.57 |
| 123 | 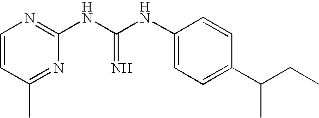 | 1-(4-sec- butylphenyl)-3-(4- methylpyrimidin-2- yl)guanidine | 284 | 1.16 |
| 124 | 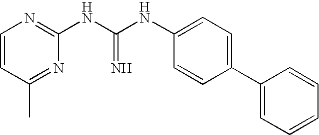 | 1-(4-phenyl-phenyl)- 3-(4- methylpyrimidin-2- yl)guanidine | 304 | 1.1 |
| 125 | 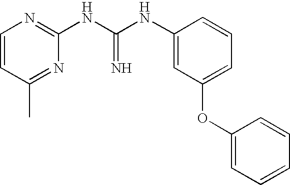 | 1-(4- methylpyrimidin-2- yl)-3-(3- phenoxyphenyl) guanidine | 320 | 1.09 |
| 126 | 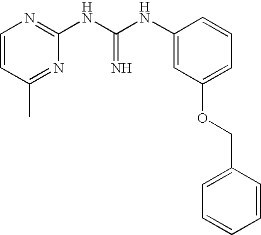 | 1-(3- (benzyloxy)phenyl)- methylpyrimidin-2- yl)guanidine | 334 | 1.13 |
| 127 | 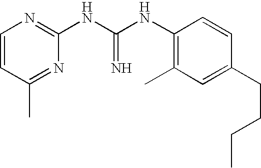 | 1-(4-butyl-2- methylphenyl)-3-(4- methylpyrimidin-2- yl)guanidine | 298 | 1.27 |
| 128 | 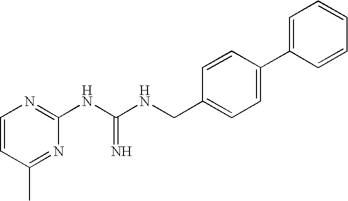 | 1-(4-phenyl-benzyl)- 3-(4- methylpyrimidin-2- yl)guanidine | 318 | 1.16 |
| 129 | 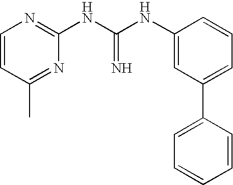 | 1-(3-ethylphenyl)-3- (4-methylpyrimidin- 2-yl)guanidine | 304 | 1.08 |
| 130 | 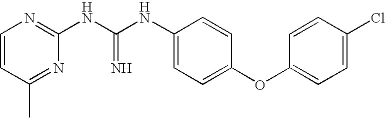 | 1-(4-(4- chlorophenoxy)phenyl)- 3-(4- methylpyrimidin-2- yl)guanidine | 354 | 1.27 |
| 131 | 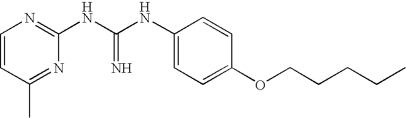 | 1-(4- methylpyrimidin-2- yl)-3-(4- (pentyloxy)phenyl) guanidine | 314 | 1.27 |
| 132 | 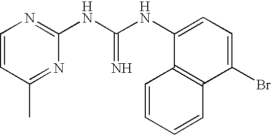 | 1-(4- bromonaphthalen-1- yl)-3-(4- methylpyrimidin-2- yl)guanidine | 357 | 1.09 |
| 133 | 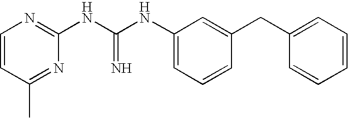 | 1-(3-benzylphenyl)- 3-(4- methylpyrimidin-2- yl)guanidine | 318 | 1.15 |
| 134 | 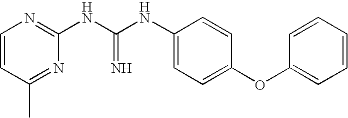 | 1-(4- methylpyrimidin-2- yl)-3-(4- phenoxyphenyl) guanidine | 320 | 1.11 |
| 135 | 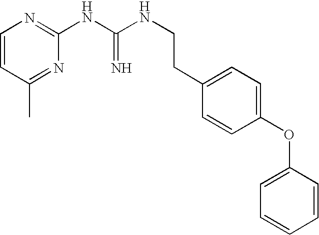 | 1-(4- phenoxyphenethyl)- 3-(4- methylpyrimidin-2- yl)guanidine | 348 | 1.23 |
| 136 | 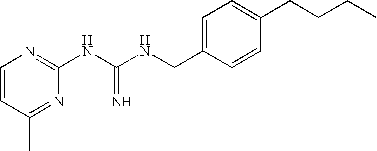 | 1-(4-butylbenzyl)-3- (4-methylpyrimidin- 2-yl)guanidine | 298 | 1.26 |
| 137 | 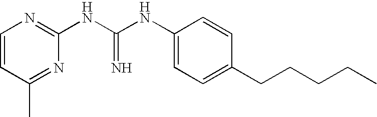 | 1-(4- methylpyrimidin-2- yl)-3-(4- pentylphenyl)guanidine | 298 | 1.33 |
| 138 | 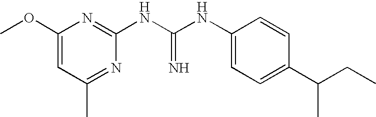 | 1-(4-sec- butylphenyl)-3-(4- methoxy-6- methylpyrimidine-2- yl)guanidine | 314 | 1.32 |
| 139 | 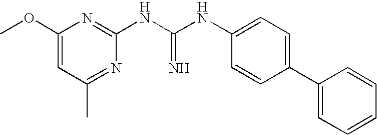 | 1-(4-phenyl-phenyl)- 3-(4-methoxy-6- methylpyrimidin-2- yl)guanidine | 334 | 1.26 |
| 140 | 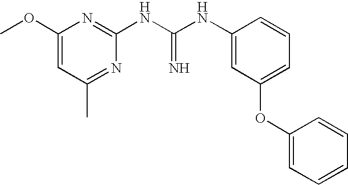 | 1-(4-methoxy-6- methylpyrimidin-2- yl)-3-(3- phenoxyphenyl) guanidine | 350 | 1.26 |
| 141 | 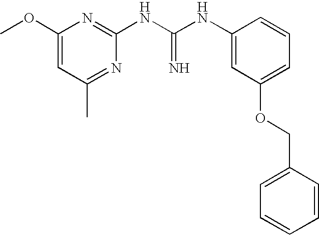 | 1-(3- (benzyloxy)phenyl) 3-(4-methoxy-6- methylpyrimidin-2- yl)guanidine | 364 | 1.3 |
| 142 | 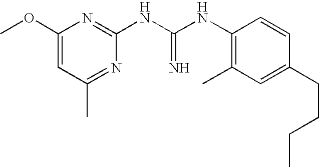 | 1-(4-butyl-2- methylphenyl)-3-(4- methoxy-6- methylpyrimidin-2- yl)guanidine | 328 | 1.44 |
| 143 | 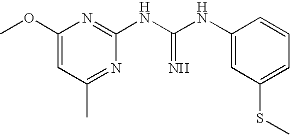 | 1-(4-methoxy-6- methylpyrimidin-2- yl)-3-(3- (methylthio)phenyl) guanidine | 304 | 1.04 |
| 144 | 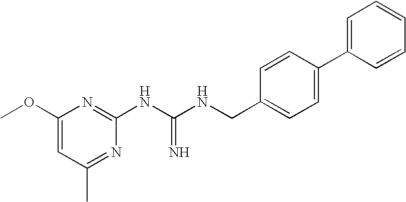 | 1-(4-phenyl-benzyl)- 3-(4-methoxy-6- methylpyrimidin-2- yl)guanidine | 348 | 1.3 |
| 145 | 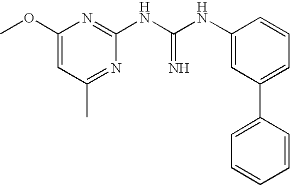 | 1-(3-phenyl-phenyl)- 3-(4-methoxy-6- methylpyrimidin-2- yl)guanidine | 334 | 1.25 |
| 146 | 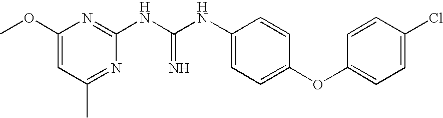 | 1-(4-(4- chlorophenoxy)phenyl)- 3-(4-methoxy-6- methylpyrimidin-2- yl)guanidine | 384 | 1.4 |
| 147 | 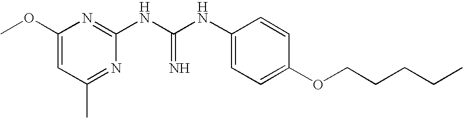 | 1-(4-methoxy-6- methylpyrimidin-2- yl)-3-(4- (pentyloxy)phenyl) guanidine | 344 | 1.41 |
| 148 | 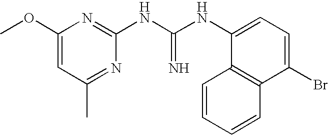 | 1-(4- bromonaphthalen-1- yl)-3-(4-methoxy-6- methylpyrimidin-2- yl)guanidine | 387 | 1.27 |
| 149 | 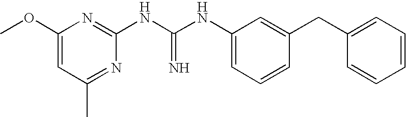 | 1-(3-benzylphenyl)- 3-(4-methoxy-6- methylpyrimidin-2- yl)guanidine | 348 | 1.29 |
| 150 | 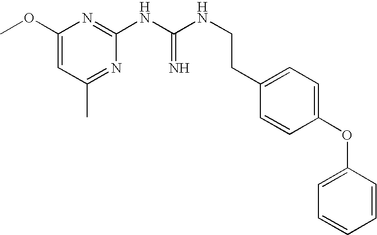 | 1-(4- phenoxyphenethyl)- 3-(4-methoxy-6- methylpyrimmdin-2- yl)guanidine | 378 | 1.36 |
| 151 | 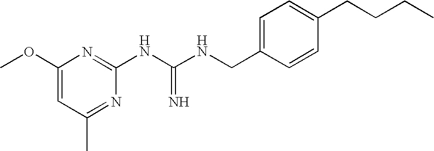 | 1-(4-butylbenzyl)-3- (4-methoxy-6- methylpyrimidin-2- yl)guanidine | 328 | 1.43 |
| 152 | 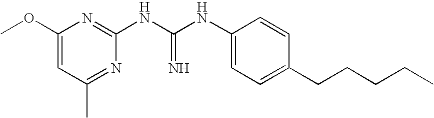 | 1-(4-methoxy-6- methylpyrimidin-2- yl)-3-(4- pentylphenyl)guanidin | 328 | 1.49 |
| 153 | 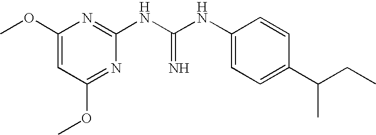 | 1-(4-sec- butylphenyl)-3-(4,6- dimethoxypyrimidin- 2-yl)guanidine | 330 | 1.32 |
| 154 | 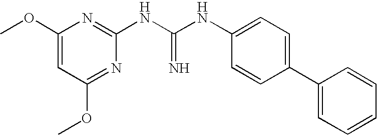 | 1-(4,6- dimethoxypyrimidin 2-yl)-3-(4-phenyl- phenyl)guanidine | 350 | 1.25 |
| 155 | 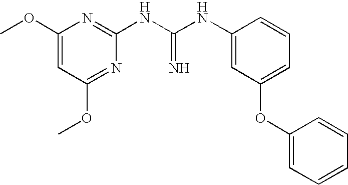 | 1-(4,6- dimethoxypyrimidin 2-yl)-3-(3- phenoxyphenyl) guanidine | 366 | 1.25 |
| 156 | 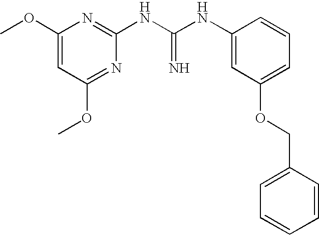 | 1-(3- (benzyloxy)phenyl)- 3-(4,6- dimethoxypyrimidin- 2-yl)guanidine | 380 | 1.29 |
| 157 | 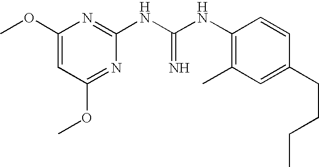 | 1-(4-butyl-2- methylphenyl)-3- (4,6- dimethoxypyrimidin- 2-yl)guanidine | 344 | 1.41 |
| 158 | 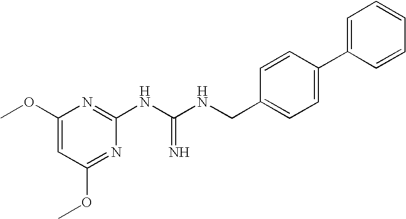 | 1-(4-phenylbenzyl)- 3-(4,6- dimethoxypyrimidin- 2-yl)guanidine | 364 | 1.31 |
| 159 | 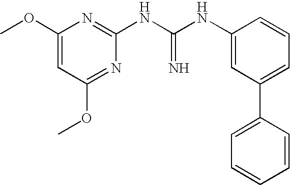 | 1-(4,6- dimethoxypyrimidin 2-yl)-3-(3-phenyl phenyl)guanidine | 350 | 1.25 |
| 160 | 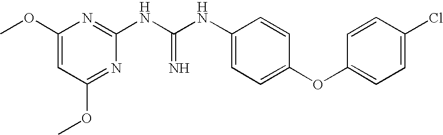 | 1-(4-(4- chlorophenoxy)phenyl)- 3-(4,6- dimethoxypyrimidin 2-yl)guanidine | 400 | 1.41 |
| 161 | 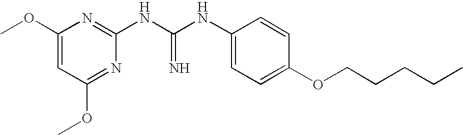 | 1-(4,6- dimethoxypyrimidin 2-yl)-3-(4- (pentyloxy)phenyl) guanidine | 360 | 1.41 |
| 162 | 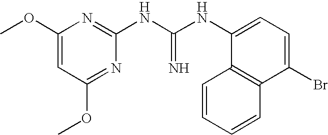 | 1-(4- bromonaphthalen-1- yl)-3-(4,6- dimethoxypyrimidin- 2-yl)guanidine | 403 | 1.26 |
| 163 | 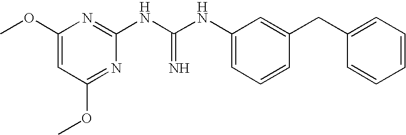 | 1-(3-benzylphenyl)- 3-(4,6- dimethoxypyrimidin 2-yl)guanidine | 364 | 1.31 |
| 164 | 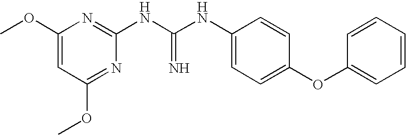 | 1-(4,6- dimethoxypyrimidin 2-yl)-3-(4- phenoxyphenyl) guanidin | 366 | 1.27 |
| 165 | 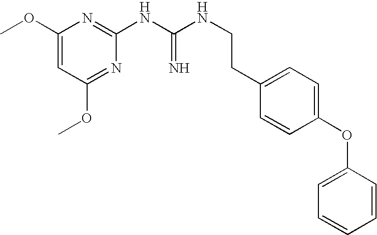 | 1-(4- phenoxyphenethyl)- 3-(4,6- dimethoxypyrimidin- 2-yl)guanidine | 394 | 1.35 |
| 166 | 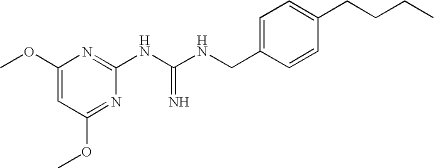 | 1-(4-butylbenzyl)-3- (4,6- dimethoxypyrimidin- 2-yl)guanidine | 344 | 1.45 |
| 167 | 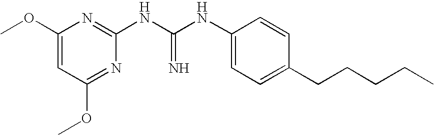 | 1-(4,6- dimethoxypyrimidin- 2-yl)-3-(4- pentylphenyl) guanidine | 344 | 1.46 |
| 168 | 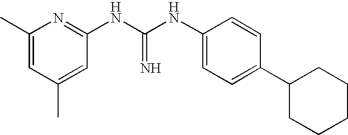 | 1-(4- cyclohexylphenyl)-3- (4,6-dimethylpyridin- 2-yl)guanidine | 323 | 1.71 |
| 169 | 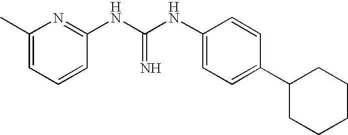 | 1-(4- cyclohexylphenyl)-3- (6-methylpyridin-2- yl)guanidine | 309 | 1.58 |
| 170 | 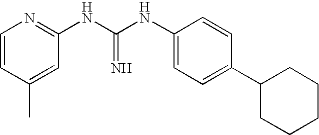 | 1-(4- cyclohexylphenyl)-3- (4-methylpyridin-2- yl)guanidine | 309 | 1.57 |
| 171 | 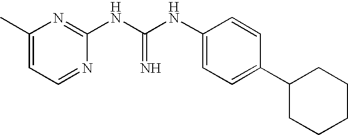 | 1-(4- cyclohexylphenyl)-3- (4-methylpyrimidin- 2-yl)guanidine | 310 | 1.32 |
| 172 | 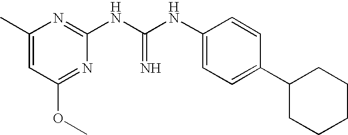 | 1-(4- cyclohexylphenyl)-3- (4-methoxy-6- methylpyrimidin-2- yl)guanidine | 340 | 1.45 |
| 173 | 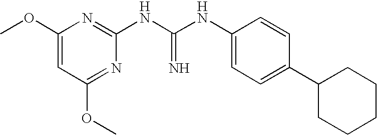 | 1-(4- cyclohexylphenyl)-3- (4,6- dimethoxypyrimidin 2-yl)guanidine | 356 | 1.45 |
| 174 | 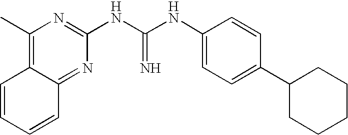 | 1-(4- cyclohexylphenyl)-3- (4-methyiquinazolin- 2-yl)guanidine | 360 | 1.54 |
| 175 | 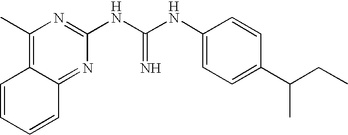 | 1-(4-sec- butylphenyl)-3-(4- methylquinazolin-2- yl)guanidine | 334 | 2.26 |
| 176 | 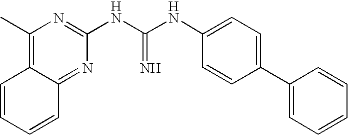 | 1-(4-phenyl-phenyl)- 3-(4- methylquinazolin-2- yl)guanidine | 354 | 2.19 |
| 177 | 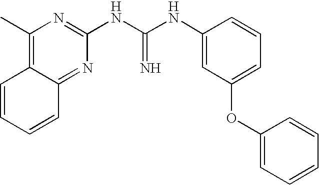 | 1-(4- methylquinazolin-2- yl)-3-(3- phenoxyphenyl)guanidine | 370 | 2.19 |
| 178 | 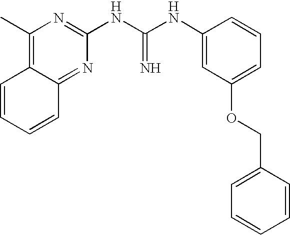 | 1-(3- (benzyloxy)phenyl) 3-(4- methylquinazolin-2- yl)guanidine | 384 | 2.21 |
| 179 | 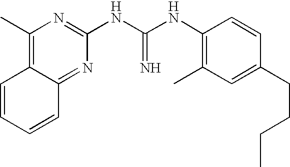 | 1-(4-butyl-2- methylphenyl)-3-(4- methylquinazolin-2- yl)guanidine | 348 | 2.34 |
| 180 | 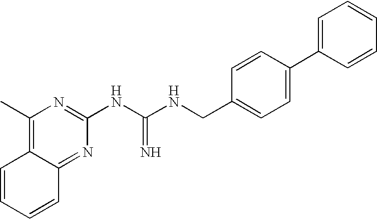 | 1-(4-phenyl-benzyl)- 3-(4- methylquinazolin-2- yl)guanidine | 368 | 2.22 |
| 181 | 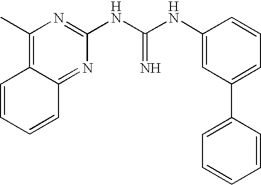 | 1-(3-phenyl-phenyl)- 3-(4- methylquinazolin-2- yl)guanidine | 354 | 2.18 |
| 182 | 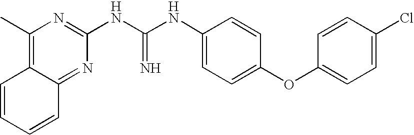 | 1-(4-(4- chlorophenoxy)phenyl)- 3-(4- methylquinazolin-2- yl)guanidine | 404 | 2.3 |
| 183 | 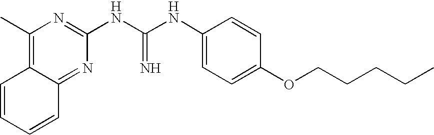 | 1-(4- methylquinazolin-2- yl)-3-(4- (pentyloxy)phenyl) guanidine | 364 | 2.33 |
| 184 | 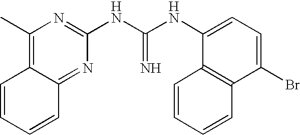 | 1-(4- bromonaplithalen-1- yl)-3-(4- methylquinazolin-2- yl)guanidine | 4.7 | 2.21 |
| 185 | 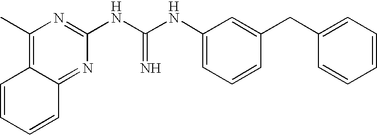 | 1-(3-benzylphenyl)- 3-(4- methylquinazolin-2- yl)guanidine | 368 | 2.23 |
| 186 | 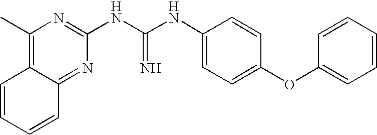 | 1-(4- methylquinazolin-2- yl)-3-)4- phenoxyphenyl) guanidine | 370 | 2.19 |
| 187 | 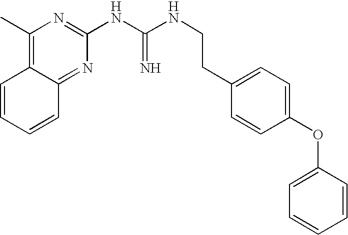 | 1-(4- phenoxyphenethyl)- 3-(4- methylquinazolin-2- yl)guanidine | 398 | 2.26 |
| 188 | 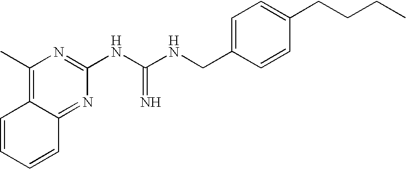 | 1-(4-butylbenzyl)-3- (4-methyiquinazolin- 2-yl)guanidine | 348 | 2.32 |
| 189 | 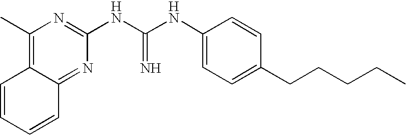 | 1-(4- methylquinazolin-2- yl)-3-(4- pentylphenyl)guanidine | 348 | 2.38 |
| 190 | 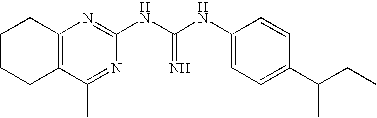 | 1-(4-sec- butylphenyl)-3-(4- methyl-5,6,7,8- tetrahydroquinazolin- 2-yl)guanidine | 338 | 2.34 |
| 191 | 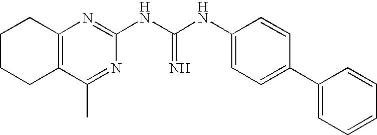 | 1-(4-phenyl-phenyl)- 3-(4-methyl-5,6,7,8- tetrahydroquinazolin- 2-yl)guanidine | 358 | 2.25 |
| 192 | 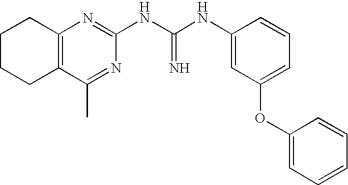 | 1-(4-methyl-5,6,7,8- tetrahydroquinazolin- 2-yl)-3-(3- phenoxyphenyl) guanidine | 374 | 2.25 |
| 193 | 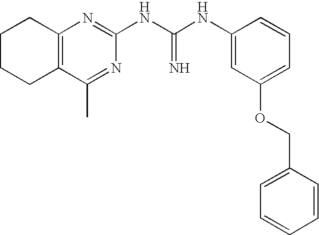 | 1-(3- (benzyloxy)phenyl)- 3-(4-methyl-5,6,7,8- tetraiiydroquinazolin- 2-yl)guanidine | 388 | 2.28 |
| 194 | 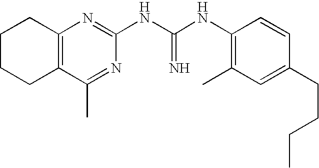 | 1-(4-butyl-2- methylphenyl)-3-(4- methyl-5,6,7,8- tetrahydroquinazolin- 2-yl)guanidine | 352 | 2.44 |
| 195 | 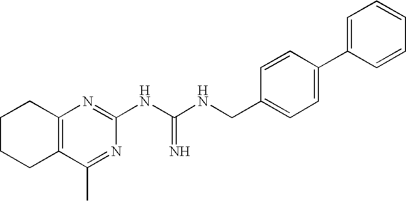 | 1-(4-phenyl-benzyl)- 3-(4-methyl-5,6,7,8- tetrahydroquinazolin- 2-yl)guanidine | 372 | 2.28 |
| 196 | 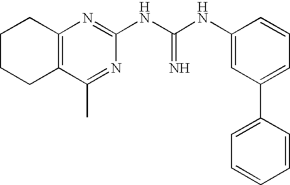 | 1-(3-phenyl-phenyl)- 3-(4-methyl-5,6,7,8- tetrahydroquinazolin- 2-yl)guanidine | 358 | 2.26 |
| 197 | 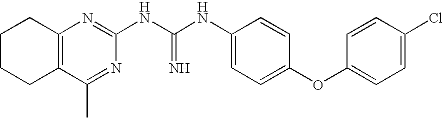 | 1-(4-(4- chlorophenoxy)phenyl)- 3-(4-methyl- 5,6,7,8- tetrahydroquinazolin- 2-yl)guanidine | 408 | 2.37 |
| 198 | 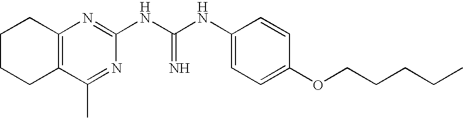 | 1-(4-methyl-5,6,7,8- tetrahydroquinazolin- 2-yl)-3-(4- (pentyloxy)phenyl) guanidine | 368 | 2.4 |
| 199 | 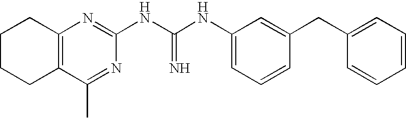 | 1-(3-benzylphenyl)- 3-(4-methyl-5,6,7,8- tetrahydroquinazolin- 2-yl)guanidine | 372 | 2.28 |
| 200 | 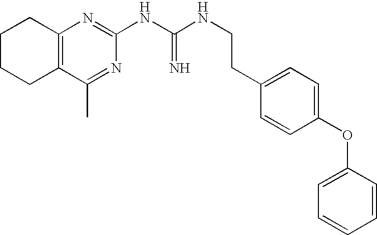 | 1-(4- phenoxyphenethyl)- 3-(4-methyl-5,6,7,8- tetrahydroquinazolin- 2-yl)guanidine | 402 | 2.32 |
| 201 | 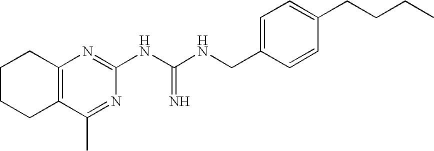 | 1-(4-butylbenzyl)-3- (4-methyl-5,6,7,8- tetrahydroquinazolin- 2-yl)guanidine | 352 | 2.42 |
| 202 | 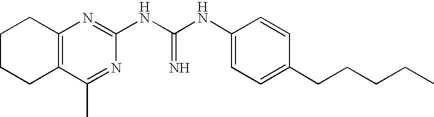 | 1-(4-methyl-5,6,7,8- tetrahydroquinazolin- 2-yl)-3-(4- pentylphenyl)guanidine | 352 | 2.48 |
| 203 | 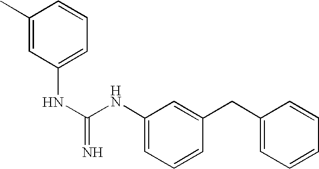 | 1-(3-benzylphenyl)- 3-m-tolylguanidine | 316 | 1.25 |
| 204 | 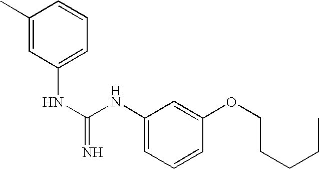 | 1-(3- (pentyloxy)phenyl)- 3-m-tolylguanidine | 312 | 1.35 |
| 205 | 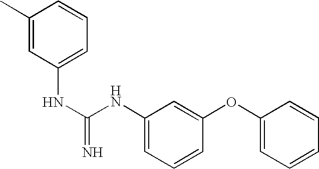 | 1-(3- phenoxyphenyl)-3- m-tolylguanidine | | |
| 206 | 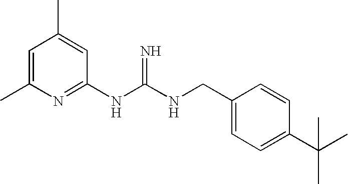 | 1-(4-tert- butylbenzyl)-3-(4,6- dimethylpyridin-2- yl)guanidine | 311 | 2.97 |
| 207 | 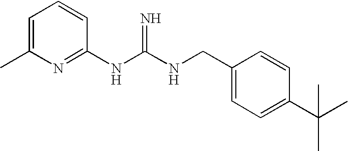 | 1-(4-tert- butylbenzyl)-3-(6- methylpyridin-2- yl)guanidine | 297 | 2.75 |
| 208 | 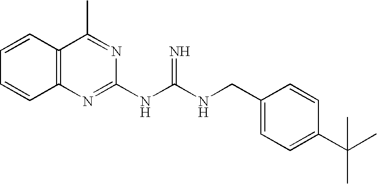 | 1-(4-tert- butylbenzyl)-3-(4- methylquinazolin-2- yl)guanidine | 348 | 2.75 |
| 209 | 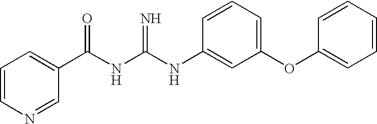 | 1-nicotinoyl-3-(3- phenoxyphenyl) guanidine | | |
| 210 | 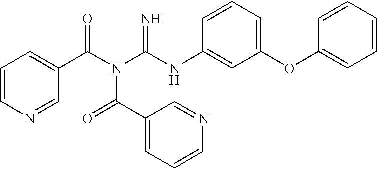 | 1,1-dinicotinoyl-3- (3-phenoxyphenyl) guanidine | | |
| 211 | 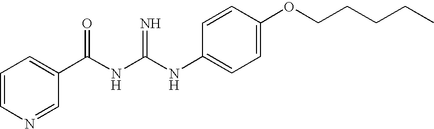 | 1-nicotinoyl-3-(4- (pentyloxy)phenyl) guanidine | | |
| 212 | 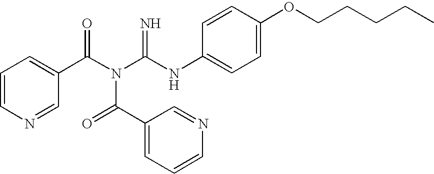 | 1,1-dinicotinoyl-3- (4-(pentyloxy) phenyl)guanidine | | |

### Example 3

### Assay for Identifying Compounds which Inhibit HCV Replication

Compounds claimed herein are tested for the ability to inhibit viral replication of the Hepatitis C replicon in cultured cells in which the HCV replicon construct has been incorporated. The HCV replicon system was described by Bartenschlager, et. al (Science, 285, pp. 110-113 (1999)). The replicon system is predictive of in vivo anti-HCV activity; compounds that are active in humans uniformly evidence activity in the replicon assay.

In this assay HCV replicon containing cells are treated with different concentrations of the test compound to ascertain the ability of the test compound to suppress replication of the HCV replicon. As a positive control, HCV replicon-containing cells are treated with different concentrations of interferon alpha, a known inhibitor of HCV replication. The replicon assay system includes Neomycin Phosphotransferase (NPT) as a component of the replicon itself in order to detect the transcription of replicon gene products in the host cell. Cells in which the HCV replicon is actively replicating have high levels of NPT; the level of NPT is proportional to HCV replication. Cells in which the HCV replicon is not replicating also have low levels of NPT and thus do not survive when treated with Neomycin. The NPT level of each sample is measured using a captured ELISA.

A protocol for testing compounds for the ability to inhibit viral replication of the Hepatitis C replicon cultured cells in which the replicon construct has been incorporated, follows.

3A. HCV Replicon and Replicon Expression

The HCV genome consists of a single ORF that encodes a 3000 amino acid polyprotein. The ORF is flanked on the 5′ side by an untranslated region that serves as an internal ribosome entry site (IRES) and at the 3′ side by a highly conserved sequence necessary for viral replication (3′-NTR). The structural proteins, necessary for viral infection, are located near the 5′ end of the ORF. The non-structural proteins, designated NS2 to NS5B comprise the remainder of the ORF.

The HCV replicon contains, 5′-3′, the HCV-IRES, the neomycin phosphotransferase (neo) gene, the IRES of encephalomyocarditis virus, which directs translation of HCV sequences NS3 to NS5B, and the 3′-NTR. The sequence of the HCV replicon has been deposited in GenBank (Accession no. AJ242652).

The replicon is transfected into Huh-7 cells using standard methods such as electroporation.

3B. Cell Maintenance

The equipment and materials include, but are not limited to, Huh-7 HCV replicon-containing cells, maintenance media (DMEM (Dulbecco's modified Eagle media) supplemented with 10% FBS, L-glutamine, non-essential amino acids, penicillin (100 units/ml), streptomycin (100 micrograms/ml), and 500 micrograms/ml of Geneticin (G418), screening media (DMEM supplemented with 10% FBS, L-glutamine, non-essential amino acids, penicillin (100 units/ml) and streptomycin (100 micrograms/ml)), 96 well tissue culture plates (flat bottom), 96 well plates (U bottom for drug dilution), Interferon alpha for positive control, fixation reagent (such as methanol: acetone), primary antibody (rabbit anti-NPTII), secondary antibody: Eu-N1 I , and enhancement solution.

HCV replicon-containing cells support high levels of viral RNA replicon replication when their density is suitable. Over-confluency causes decreased viral RNA replication. Therefore, cells must be kept growing in log phase in the presence of 500 micrograms/ml of G418. Generally, cells should be passed twice a week at 1: 4-6 dilution. Cell maintenance is conducted as follows:

HCV replicon-containing cells are examined under a microscope to ensure that cells growing well. Cells are rinsed once with PBS and 2 ml trypsin is added. The cell/trypsin mixture is incubated at 37° C. in a CO₂ incubator for 3-5 minutes. After incubation 10 ml of complete media is added to stop the trypsinization reaction. Cells are blown gently, put into a 15 ml tube, and spun at 1200 rpm for 4 minutes. The trypsin/medium solution is removed. Medium (5 ml) is added and the cells are mixed carefully. The cells are counted.

The cells are then seeded onto 96-well plates at a density of 6000-7500 cells/100 microliters/well (6-7.5×105 cells/10 ml/plate). The plates are then incubated at 37° C. in a 5% CO₂ incubator.

Cells are examined under a microscope approximated 24 hours after seeding and prior to adding drugs. If counting and dilution were performed correctly, cells are 60-70% confluent and nearly all cells should attach and spread evenly in the well.

3C. Treatment of HCV-Replicon Containing Cells with Test Compound

HCV replicon-containing cells are rinsed with once PBS once; 2 mls of trypsin are then added. Cells are incubated at 37° C. in a 5% CO₂ incubator for 3-5 minutes. 10 mls of complete medium is added to stop the reaction. Cells are blown gently, put into a 15 ml tube, and spun at 1200 rpm for four minutes. The trypsin/medium solution is removed and 5 mls of medium (500 ml DMEM (high glucose)) from BRL catalog #12430-054; 50 mls 10% FBS, 5% Geneticin G418 (50 mg/ml, BRL catalog #10131-035), 5 ml MEM non-essential amino acids (100×BRL #11140-050) and 5 ml pen-strep (BRL #15140-148) is added. The cells and media are mixed carefully

Cells are plated with screening medium (500 ml DMEM (BRL #21063-029), 50 ml FBS (BRL #10082-147) and 5 ml MEM non-essential amino acid (BRL #11140-050) at 6000-7500 cells/100 μl/well of 96 well plate (6-7.5×105 cells/10 ml/plate). Plates are placed into 37° C. 5% CO₂ incubator overnight.

3D. Assay

The following morning, drugs (test compounds or interferon alpha) are diluted in 96 well U bottom plates with media or DMSO/media, depending on the final concentration chosen for screening. Generally for 6 concentrations of each test compounds ranging from 10 micromolar to 0.03 micromolar are applied. 100 μl of the test compound dilution is placed in wells of the 96 well plate containing the HCV replicon cells. Media without drug is added to some wells as a negative controls. DMSO is known to affect cell growth. Therefore, if drugs diluted in DMSO are used, all wells, including negative control (media only) and positive control (interferon alpha) wells, must contain the same concentration of DMSO, for single dose screening. The plates are incubated at 37° C. in a humidified 5% CO₂ environment for three days.

On day four, the NTPII assay is quantitated. The medium is poured from the plates and the plates are washed once in 200 μl of PBS. The PBS is then decanted and the plates tapped in a paper towel to remove any remaining PBS. Cells are fixed in situ with 100 μl/well of pre-cooled (−20° C.) methanol: acetone (1:1) and the plates are placed at −20° C. for 30 minutes.

The fixing solution is poured from the plates and the plates allowed to air-dry completely (approximately one hour). The appearance of the dried cell layer is recorded and the density of the cells in the toxic wells is scored with the naked eye. Alternatively cell viability may be assessed using the MTS assay described below.

The wells are blocked with 200 pt of blocking solution (10% FBS; 3% NGS in PBS) for 30 minutes at room temperature. The blocking solution is removed and 100 μl of rabbit anti-NPTII diluted 1:1000 in blocking solution is added to each well. The plates are then incubated 45-60 minutes at room temperature. After incubation, wells are washed six times with PBS-0.05% Tween-20 solution. 100 μl of 1:15,000 diluted Europium (EU)-conjugated goat anti-rabbit in blocking buffer is added to each well and incubated at room temperature for 30-45 minutes. The plates are washed again and 100 μl of enhancement solution (Perkin Elmer #4001-0010) is added to each well. Each plate is shaken (approx. 30 rpm) in a plate shaker for three minutes. 95 μl is transferred from each well to a black plate; the EU signal is quantitated in a Perkin-Elmer VICTOR plate reader (EU-Lance).

Test Results:

Compounds 1-208 disclosed in Table I have been tested in the above assay and found to inhibit replication of the HCV replicon with EC₅₀ values of less than 5 micromolar.

### Example 4

### Cytotoxicity Assays

To insure that the decrease in replicon replication is due to compound activity against the HCV replicon rather than nonspecific toxicity assays are used to quantitate compound cytotoxicity.

### Example 4A

### Cellular Protein Albumin Assay for Cytotoxicity

Cellular protein albumin measurements provide one marker of cytotoxicity. The protein levels obtained from cellular albumin assays may also be used to provide a normalization reference for antiviral activity of compounds. In the protein albumin assay HCV replicon-containing cells are treated for three days with different concentrations of helioxanthin; a compound that is known to be cytotoxic at high concentrations. The cells are lysed and the cell lysate used to bind plate-bound goat anti-albumin antibody at room temperature (25° C. to 28° C.) for 3 hours. The plate is then washed 6 times with 1×PBS. After washing away the unbound proteins, mouse monoclonal anti-human serum albumin is applied to bind the albumin on the plate. The complex is then detected using phosphatase-labeled anti-mouse IgG as a second antibody.

### Example 4B

### MTS Assay for Cytotoxicity

Cell viability may also be determined by CELLTITER 96 AQUEOUS ONE Solution Cell Proliferation Assay (Promega, Madison Wis.), a calorimetric assay for determining the number of viable cells. In this method, before fixing the cells, 10-20 μl MTS reagent is added to each well according to manufacturer's instructions, plates are incubated at 37° C. and read at OD 490 nm. During the incubation period living cells covert the MTS reagent to a formazan product which absorbs at 490 nm. Thus the 490 nm absorbance is directly proportional to the number of living cells in culture.

A direct comparison of the Cellular Albumin and MTS methods for determining cytotoxicity may be obtained as follows: Cells are treated with different concentrations of test compound or Helioxanthin for a three day-period. Prior to lysis for detection albumin as described above, the MTS reagent is added according to manufacturer's instruction to each well and incubate at 37° C. and read at OD 490 nm. The cellular albumin quantitation is then performed as described above.

### Example 5

### Pharmaceutical Formulations

Examples 5A through 5G are examples of pharmaceutical compositions containing the compounds of Formula 1. The abbreviation “V.I.” stands for the viral inhibitor compounds of Formula 1 of the present invention.

### Example 5A

### Oral Drops

5 grams of V.I. is dissolved in 5 ml of 2-hydroxypropanoic acid and 15 ml polyethylene glycol at about 60° C. to about 80° C. After cooling to about 30°-40° C., 350 ml polyethylene glycol is added and the mixture was stirred well. A solution of 17.5 g sodium saccharin in 25 ml purified water is then added. Flavor and polyethylene glycol q.s. (quantity sufficient) to a volume of 500 ml are added while stirring to provide an oral drop solution comprising 10 mg/ml of V.I.

### Example 5B

### Capsules

20 grams of the V.I., 6 grams sodium lauryl sulfate, 56 grams starch, 56 grams lactose, 0.8 grams colloidal silicon dioxide, and 1.2 grams magnesium stearate are vigorously stirred together. The resulting mixture is subsequently filled into 1000 suitable hardened gelatin capsules, comprising each 20 mg of the active ingredient.

### Example 5C

### Film-Coated Tablets

Preparation of tablet core: A mixture of 10 grams of the V.I., 57 grams lactose and 20 grams starch is mixed well and thereafter humidified with a solution of 0.5 grams sodium dodecyl sulfate, and 1.0 grams polyvinylpyrrolidone (KOLLIDON-K 90) in about 20 ml of water. The wet powder mixture is sieved, dried, and sieved again. Then 100 grams microcrystalline cellulose (AVICEL) and 15 grams hydrogenated vegetable oil (STEROTEX) are added. The whole is mixed well and compressed into tablets, giving 1000 tablets, each containing 10 mg of the active ingredient.

Coating: Ethyl cellulose (0.5 grams, ETHOCEL 22 CPS) in 15 ml of dichloromethane is added to a solution of 1.0 grams methyl cellulose (Methocel 60 HG.RTM.) in 7.5 ml of denatured ethanol. Then 7.5 ml of dichloromethane and 0.25 ml 1,2,3-propanetriol are added. Polyethylene glycol (1.0 grams) is melted and dissolved in 7.5 ml of dichloromethane and added to the cellulose-containing solution. Magnesium Octadecanoate (0.25 grams), 0.5 grams polyvinylpyrrolidone, and 3.0 ml of concentrated color suspension (OPASPRAY K-1-2109) are added and the whole mixture homogenized. The tablet cores are coated with this mixture in a coating apparatus.

### Example 5D

### Injectible Solutions

(i) 1.8 grams methyl 4-hydroxybenzoate and 0.2 grams propyl 4-hydroxybenzoate are dissolved in about 0.5 L of boiling water. After cooling to about 50° C., 4 grams lactic acid, 0.05 grams propylene glycol, and 4 grams of viral inhibitor are added while stirring. The solution is cooled to room temperature and supplemented with water for injection q.s. giving a solution containing 4 mg/ml of V.I. The solution is sterilized by filtration and filled in sterile containers.

(ii) 100.0 g of an acid salt of an V.I. of the invention is dissolved in boiling water. After cooling to about 50° C., 37.5 grams lactic acid (90% by weight) are added while stirring. The solution is cooled to room temperatutre and water is added to 1 L. The solution is sterilized by filtration and filled in sterile containers.

(iii) 5.00 g of an acid salt of an V.I. of the invention is dissolved in boiling water. After cooling to about 50° C., 2.20 grams lactic acid (90% by weight) are added while stirring. The solution is cooled to room temperature and water is added to 100 ml.

### Example 5E

### Gel

A compound or salt of the invention may be formed as a gel for topical application.

A gel is prepared by suspending V.I. (0.2 g-5.0 g) in benzyl alcohol at room temperature. A mixture of hydroxypropyl cellulose (2.5) grams and demineralized water (q.s. 100 g) is added to the suspension with stirring.

### Example 5F

### Cream

Phase I contains Sorbitan monostearate (2.0 g), Polyoxyethylene (20) sorbitan monostearate (1.5 g), Synthetic spermaceti (3.0 g) Cetyl stearyl alcohol (10.0 g) and 2-Octyldodecanol (13.5 g). The phase I mixture is heated to 75° C., stirred and mixed.

Phase II contains V.I. (1.0 g). Phase II is added to phase I, stirred and suspended.

Phase III contains Benzyl alcohol (1.0 g) and demineralized water (q.s. 100 g). Phase III is heated to 75° C. and added to phase II. The cream is mixed intensively and cooled slowly to room temperature, with further stirring. After cooling to room temperature the cream is homogenized.

### Example 5G

### Sprays

The active compound solutions or suspensions prepared according to Example 5D can also be processed to sprays. For this purpose, for example, a 60 to 90% active compound solution is mixed with 20 to 40% of the usual propellants, for example N₂, N₂O, CO₂, propane, butane, halogenohydrocarbons and the like.

1. A compound of the formula 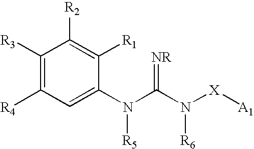
or a pharmaceutically acceptable salt thereof, wherein:
X is absent, —CR′R″—, —(CR′R″)₂—, —CR′R″O—, —O—, or NR;
Each R is independently hydrogen, C₁-C₆alkyl, or cyano;
R′ and R″ are independently hydrogen, halogen, C₁-C₂alkyl, or C₁-C₂alkoxy;
R₁, R₂, R₃, and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); where at least one of R₁, R₂, R₃, or R₄ is not hydrogen; or
any two of R₁, R₂, R₃ and R₄ which are bound to adjacent carbon atoms are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino, and
the remaining two of R₁, R₂, R₃, and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); where at least one of R₁, R₂, R₃, or R₄ is not hydrogen;
R₅ and R₆ are independently hydrogen, C₁-C₄alkyl, C₂-C₄alkenyl, or C₃-C₈cycloalkyl(C₀-C₂alkyl);
A₁ is
i) phenyl, which is substituted with 1 LA₂ substituent and 0 or 1 or more R₇ substituents; or
ii) naphthyl, indanyl, or 9H-fluorenyl, each of which is substituted with 0 or 1 LA₂ substituent, and 0 or 1 or more R₇ substituents; or
iii) heteroaryl, which is substituted with 0 or 1 LA₂ substituent, and 0 or 1 or more R₇ substituents;
R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy);
L is absent, —CR₈R₉—, —(CR₈R₉)₂—, —CR₈R₉O—, —OCR₈R₉—, —O(CR₈R₉)₂—, —NH(C═O)—, —NH(SO₂)—, —O—, or NR₁₀; where
R₈ and R₉ are independently hydrogen, halogen, C₁-C₂alkyl, or C₁-C₂alkoxy;
R₁₀ is hydrogen, C₁-C₆alkyl, or cyano; and
A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- and di-(C₁-C₆alkyl)amino, mono- and di-(C₁-C₄alkyl)amino(C₁-C₄alkyl), and mono- and di-(C₁-C₄alkyl)amino(C₁-C₄alkoxy).

2. (canceled)

3. A compound of the formula 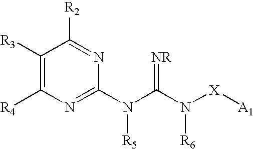
or a pharmaceutically acceptable salt thereof, wherein:
X is absent, —CR′R″—, —(CR′R″)₂—, —CR′R″O—, —O—, or NR;
Each R is independently hydrogen, C₁-C₆alkyl, or cyano;
R′ and R″ are independently hydrogen, halogen, C₁-C₂alkyl, or C₁-C₂alkoxy;
R₂ is C₁-C₂alkyl, C₁-C₂alkoxy, or trifluoromethyl;
R₃ and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); or
R₃ and R₄ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino;
R₅ and R₆ are independently hydrogen, C₁-C₄alkyl, C₂-C₄alkenyl, or C₃-C₈cycloalkyl(C₀-C₂alkyl);
A₁ is
i) phenyl, which is substituted with 1 LA₂ substituent and 0 or 1 or more R₇ substituents; or
ii) naphthyl, indanyl, or 9H-fluorenyl, each of which is substituted with 0 or 1 LA₂ substituent, and 0 or 1 or more R₇ substituents; or
iii) heteroaryl, which is substituted with 0 or 1 LA₂ substituent, and 0 or 1 or more R₇ substituents;
R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy);
L is absent, —CR₈R₉—, —(CR₈R₉)₂—, —CR₈R₉O—, —OCR₈R₉—, —O(CR₈R₉)₂—, —NH(C═O)—, or —O—, where
R₈ and R₉ are independently hydrogen, halogen, C₁-C₂alkyl, or C₁-C₂alkoxy; and
A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino, with the proviso that when R₂ and R₄ are both methyl and R₃ is hydrogen
LA₂ is not phenoxy, phenylethoxy, 5-dimethylbenzo[d]thiazolyl, allyloxy, piperidin-1-ylsulfonyl, or pentoxy.

4. (canceled)

5. A compound or salt of claim 3 wherein
X is absent, —CH₂—, or —(CH₂)₂—.

6-7. (canceled)

8. A compound or salt of claim 5 wherein
R₃ is hydrogen; and
R₄ is hydrogen, hydroxy, C₁-C₂alkyl, C₁-C₂alkoxy, or trifluoromethyl.

9-10. (canceled)

11. A compound or salt of claim 3 of the formula 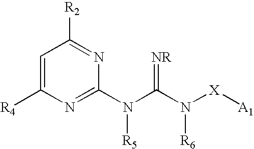
wherein
X is absent, —CH₂—, or —CH₂CH₂—;
R₂ and R₄ are independently hydrogen, methyl, and methoxy, wherein at least one of R₂ and R₄ is not hydrogen; and
R₅ and R₆ are independently hydrogen or methyl.

12-17. (canceled)

18. A compound or salt of claim 3 of the formula 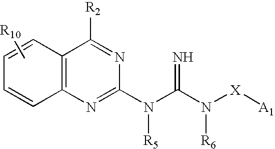
wherein
X is absent, —CH₂—, or —CH₂CH₂—;
R₂ is methyl or methoxy;
R₅ and R₆ are independently hydrogen or methyl; and
R₁₀ represents 0 to 3 substitutents independently chosen from halogen, C₁-C₂alkyl, and C₁-C₂alkoxy.

19-23. (canceled)

24. A compound or salt of claim 1 of the formula 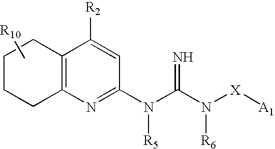
X is absent, —CH₂—, or —CH₂CH₂—;
R₂ is methyl or methoxy;
R₅ and R₆ are independently hydrogen or methyl; and
R₁₀ represents 0 to 3 substitutents independently chosen from halogen, C₁-C₂alkyl, and C₁-C₂alkoxy.

25. (canceled)

26. A compound or salt of claim 1 of the formula 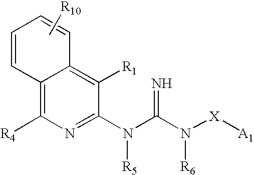
X is absent, —CH₂—, or —CH₂CH₂—;
R₁ and R₄ are independently hydrogen, methyl, or methoxy;
R₅ and R₆ are independently hydrogen or methyl; and
R₁₀ represents 0 to 3 substitutents independently chosen from halogen, C₁-C₂alkyl, and C₁-C₂alkoxy.

27. A compound or salt of claim 1 of the formula 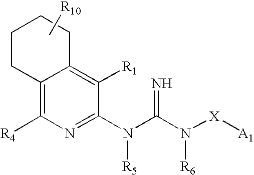
X is absent, —CH₂—, or —CH₂CH₂—
R₁ and R₄ are independently hydrogen, methyl, or methoxy;
R₅ and R₆ are independently hydrogen or methyl; and
R₁₀ represents 0 to 3 substitutents independently chosen from halogen, C₁-C₂alkyl, and C₁-C₂alkoxy.

28-31. (canceled)

32. A compound or salt of claim 3, wherein 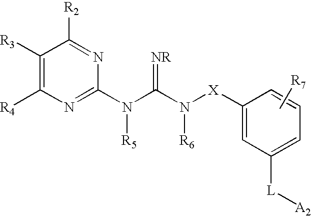
wherein
R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino;
L is absent, —CR₈R₉—, or —O—, where
R₈ and R₉ are independently hydrogen, halogen, C₁-C₂alkyl, or C₁-C₂alkoxy; and
A₂ is C₃-C₆alkyl or C₃-C₆alkenyl, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino.

33. A compound or salt of claim 3 of the formula 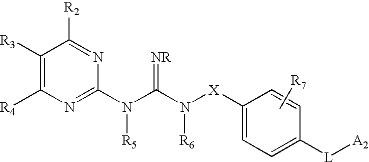
wherein
R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino;
L is absent, —CR₈R₉—, or —O—, where
R₈ and R₉ are independently hydrogen, halogen, C₁-C₂alkyl, or C₁-C₂alkoxy; and
A₂ is C₃-C₆alkyl or C₃-C₆alkenyl, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino.

34. (canceled)

35. A compound or salt of claim 3 of the formula 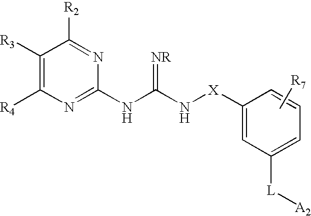
wherein
R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino;
L is absent, —CR₈R₉—, —(CR₈R₉)₂—, —CR₉R₉O—, —O(CR₈R₉)—, —O(CR₈R₉)₂—, —NH(C═O)—, or —O—, where
R₈ and R₉ are independently hydrogen, halogen, C₁-C₂alkyl, or C₁-C₂alkoxy; and
A₂ is phenyl, C₃-C₈cycloalkyl, a monocyclic or bicyclic heteroaryl group having 1, 2, or 3 heteroatoms chosen from N, O, and S, a 5- to 7-membered monocyclic heterocycloalkyl group having 1 N ring atom and 0 or 1 additional heteroatoms chosen from N, O, and S, an 8- to 12-membered bicyclic heterocycloalkyl group having 1 N ring atom and 0 to 2 additional heteroatoms chosen from N, O, and S, or a partially unsaturated monocyclic or bicyclic heterocyclic group having 1 N ring atom and 0 to 2 additional heteroatoms chosen from N, O, and S, each of which A₂ is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino.

36. A compound or salt of claim 3 of the formula 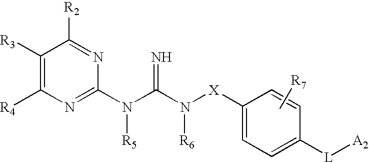
wherein
R₇ is 0 to 3 substitutents independently chosen from halogen, hydroxy, cyano, nitro, amino, acetyl, oxo, C₁-C₄alkyl, C₂-C₄alkenyl, C₁-C₄alkoxy, C₁-C₂haloalkyl, C₁-C₂haloalkoxy, and mono- and di-(C₁-C₄alkyl)amino;
L is absent, —CR₈R₉—, —(CR₈R₉)₂—, —CR₈R₉O—, —O(CR₈R₉)—, —O(CR₈R₉)₂—, —NR(C═O)—, or —O—, where
R₈ and R₉ are independently hydrogen, halogen, C₁-C₂alkyl, or C₁-C₂alkoxy; and
A₂ is phenyl, C₃-C₈cycloalkyl, a monocyclic or bicyclic heteroaryl group having 1, 2, or 3 heteroatoms chosen from N, O, and S, a 5- to 7-membered monocyclic heterocycloalkyl group having 1 N ring atom and 0 or 1 additional heteroatoms chosen from N, O, and S, an 8- to 12-membered bicyclic heterocycloalkyl group having 1 N ring atom and 0 to 2 additional heteroatoms chosen from N, O, and S, or a partially unsaturated monocyclic or bicyclic heterocyclic group having 1 N ring atom and 0 to 2 additional heteroatoms chosen from N, O, and S, each of which A₂ is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino.

37-38. (canceled)

39. A compound or salt of claim 35 of the formula 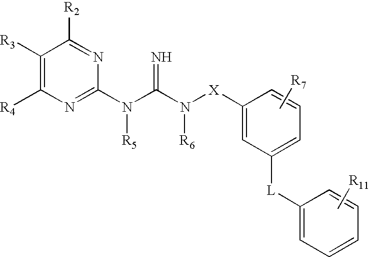
wherein
X is absent, —CH₂—, or CH₂CH₂—;
R₅ and R₆ are independently hydrogen or methyl; and
R₁₁ is 0 to 3 substituents independently chosen from halogen, hydroxy, cyano, acetyl, C₁-C₆alkyl, C₂-C₄alkenyl, C₁-C₄alkoxy, C₁-C₂haloalkyl, C₁-C₂haloalkoxy, C₁-C₄alkylthio, and mono- and di-(C₁-C₆alkyl)amino.

40. A compound or salt of claim 36 of the formula 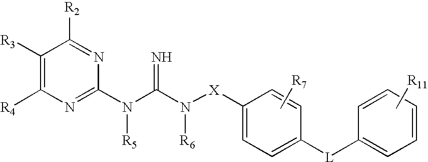
wherein
X is absent, —CH₂—, or CH₂CH₂—;
R₅ and R₆ are independently hydrogen or methyl; and
R₁₁ is 0 to 3 substituents independently chosen from halogen, hydroxy, cyano, acetyl, C₁-C₆alkyl, C₂-C₄alkenyl, C₁-C₄alkoxy, C₁-C₂haloalkyl, C₁-C₂haloalkoxy, C₁-C₄alkylthio, and mono- and di-(C₁-C₆alkyl)amino.

41. A compound or salt of claim 35 of the formula 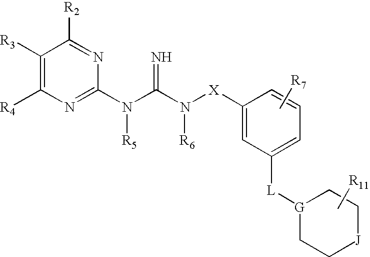
wherein
G is CH or N;
J is CH₂, N, O, or S;
X is absent, —CH₂—, or —CH₂CH₂—;
R₅ and R₆ are independently hydrogen or methyl; and
R₁₁ is 0 to 3 substituents independently chosen from halogen, hydroxy, cyano, acetyl, C₁-C₆alkyl, C₂-C₄alkenyl, C₁-C₄alkoxy, C₁-C₂haloalkyl, C₁-C₂haloalkoxy, C₁-C₄alkylthio, and mono- and di-(C₁-C₆alkyl)amino.

42. A compound or salt of claim 36 of the formula 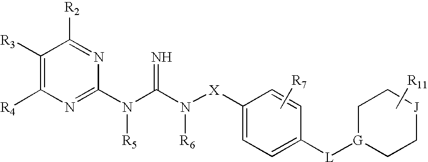
wherein
G is CH or N;
J is CH₂, N, O, or S;
X is absent, —CH₂—, or —CH₂CH₂—;
R₅ and R₆ are independently hydrogen or methyl; and
R₁₁ is 0 to 3 substituents independently chosen from halogen, hydroxy, cyano, acetyl, C₁-C₆alkyl, C₂-C₄alkenyl, C₁-C₄alkoxy, C₁-C₂haloalkyl, C₁-C₂haloalkoxy, C₁-C₄alkylthio, and mono- and di-(C₁-C₆alkyl)amino.

43-46. (canceled)

47. A compound or salt wherein the compound is
1-(4,6-dimethylpyrimidin-2-yl)-3-(4-methoxyphenyl) guanidine;
1-(4,6-dimethylpyrimidin-2-yl)-3-(3-phenoxyphenyl) guanidine;
1-(4,6-dimethylpyrimidin-2-yl)-3-(4-(pentyloxy) phenyl)guanidine;
1-(4,6-dimethylpyrimidin-2-yl)-3-(4-phenyl-phenyl)guanidine;
1-(3-phenyl-benzyl)-3-(4,6-dimethylpyrimidin-2-yl)guanidine;
1-(4,6-dimethylpyrimidin-2-yl)-3-(2-(trifluoromethyl)-1H-benzo[d]imidazol-5-yl)guanidine;
1-(4,6-dimethylpyrimidin-2-yl)-3-(2-methoxy-5-phenoxyphenyl)guanidine;
1-(4,6-dimethylpyrimidin-2-yl)-3-(3-methoxy-4-phenyl-phenyl)guanidine;
1-(4,6-dimethylpyrimidin-2-yl)-3-(1H-indazol-5-yl)guanidine;
1-(4,6-dimethylpyrimidin-2-yl)-3-(3-phenyl-4-methoxyphenyl)guanidine;
1-(4,6-dimethylpyrimidin-2-yl)-3-(2-phenoxyphenyl)guanidine;
1-(4,6-dimethylpyrimidin-2-yl)-3-(2-methylquinolin-8-yl)guanidine;
1-(4,6-dimethylpyrimidin-2-yl)-3-(quinolin-8-yl)guanidine;
1-(4,6-dimethylpyrimidin-2-yl)-3-(4-(hexyloxy) phenyl)guanidine;
1-(4,6-dimethylpyrimidin-2-yl)-3-(3-phenyl-phenyl)guanidine;
1-(4,6-dimethylpyrimidin-2-yl)-3-(4-(p-tolyloxy)phenyl)guanidine,
1-(4,6-dimethylpyrimidin-2-yl)-3-(4-(piperidin-1-yl)phenyl)guanidine;
1-(4,6-dimethylpyrimidin-2-yl)-3-(3-(2-methylpyrimidin-4-yl)phenyl)guanidine;
1-(4-sec-butylphenyl)-3-(4,6-dimethylpyrimidin-2-yl)guanidine;
1-(benzo [d]thiazol-6-yl)-3-(4,6-dimethylpyrimidin-2-yl)guanidine;
1-(4-(1H-imidazol-1-yl)phenyl)-3-(4,6-dimethylpyrimidin-2-yl)guanidine;
1-(4-butyl-2-methylphenyl)-3-(4,6-dimethylpyrimidin-2-yl)guanidine;
1-(2,3-dihydro-1H-inden-5-yl)-3-(4,6-dimethylpyrimidin-2-yl)guanidine;
1-(4,6-dimethylpyrimidin-2-yl)-3-(4-morpholino-phenyl)guanidine;
1-(3-benzylphenyl)-3-(4,6-dimethylpyrimidin-2-yl)guanidine;
1-(4-butoxyphenyl)-3-(4,6-dimethylpyrimidin-2-yl)guanidine;
1-(3-tert-butylphenyl)-3-(4,6-dimethylpyrimidin-2-yl)guanidine;
1-(4,6-dimethylpyrimidin-2-yl)-3-(2-phenyl-phenyl)guanidine;
1-(2-benzylphenyl)-3-(4,6-dimethylpyrimidin-2-yl)guanidine;
1-(4-bromonaphthalen-1-yl)-3-(4,6-dimethylpyrimidin-2-yl)guanidine;
1-(5-bromo-1H-indol-7-yl)-3-(4,6-dimethylpyrimidin-2-yl)guanidine;
1-(5-cyclohexyl-2-methoxyphenyl)-3-(4,6-dimethylpyrimidin-2-yl)guanidine;
1-(4,6-dimethylpyrimidin-2-yl)-3-(9H-fluoren-2-yl)guanidine;
1-(4,6-dimethylpyrimidin-2-yl)-3-(2-(piperidin-1-yl)phenyl)guanidine;
1-(4,6-dimethylpyrimidin-2-yl)-3-(2-methoxy-5-phenyl-phenyl)guanidine;
1-(4-(4-chlorophenoxy) phenyl)-3-(4,6-dimethylpyrimidin-2-yl)guanidine;
1-(4-benzylphenyl)-3-(4,6-dimethylpyrimidin-2-yl)guanidine;
1-(4-tert-butylphenyl)-3-(4,6-dimethylpyrimidin-2-yl)guanidine;
1-(4-cyclohexylphenyl)-3-(4,6-dimethylpyrimidin-2-yl)guanidine;
1-(4,6-dimethylpyrimidin-2-yl)-3-(4-(methoxymethyl)-2-oxo-2H-chromen-7-yl)guanidine;
1-(4,6-dimethylpyrimidin-2-yl)-3-(3-(2-hydroxyethylsulfonyl)phenyl)guanidine;
1-(4,6-dimethylpyrimidin-2-yl)-3-(2-mercaptobenzo[d]thiazol-6-yl)guanidine;
1-(4-butylbenzyl)-3-(4,6-dimethylpyrimidin-2-yl)guanidine;
1-(4-phenyl-benzyl)-3-(4,6-dimethylpyrimidin-2-yl)guanidine;
1-(4-phenoxyphenethyl)-3-(4,6-dimethylpyrimidin-2-yl)guanidine;
1-(6-methylpyridin-2-yl)-3-(4-phenoxyphenyl)guanidine;
1-(6-methylpyridin-2-yl)-3-(4-phenyl-phenyl)guanidine;
1-(4,6-dimethylpyridin-2-yl)-3-(4-phenoxyphenyl)guanidine;
1-(4,6-dimethylpyridin-2-yl)-3-(4-phenyl-phenyl)guanidine;
1-(3-(benzyloxy)phenyl)-3-(4,6-dimethylpyridin-2-yl)guanidine;
1-(4-methoxy-6-methylpyrimidin-2-yl)-3-(4-phenoxyphenyl)guanidine;
1-(4-tert-butylbenzyl)-3-(4,6-dimethylpyrimidin-2-yl)guanidine;
1-(4,6-dimethylpyrimidin-2-yl)-3-(3-fluoro-4-(pentyloxy)phenyl)guanidine;
1-(3-(indan-2-yloxy)phenyl)-3-(4,6-dimethylpyrimidin-2-yl)guanidine;
1-(4,6-dimethylpyrimidin-2-yl)-3-(3-phenethoxyphenyl)guanidine;
1-(4,6-dimethylpyrimidin-2-yl)-3-(2-phenylbenzo[d][1,3]dioxol-5-yl)guanidine;
1-(4-(3,4-dihydroisoquinolin-2(1H)-yl)-3-fluorophenyl)-3-(4,6-dimethylpyrimidin-2-yl)guanidine;
1-(4-(3,4-dihydroisoquinolin-2(1H)-yl)phenyl)-3-(4,6-dimethylpyrimidin-2-yl)guanidine;
1-(4,6-dimethylpyrimidin-2-yl)-3-(4-(octahydroisoquinolin-2(1H)-yl)phenyl)guanidine;
1-(4,6-dimethylpyrimidin-2-yl)-3-(3-fluoro-4-(octahydroquinolin-1(2H)-yl)phenyl)guanidine;
1-(3-(4-phenyl-benzyloxy)phenyl)-3-(4,6-dimethylpyrimidin-2-yl)guanidine;
1-(3-(3,4-difluorobenzyloxy) phenyl)-3-(4,6-dimethylpyrimidin-2-yl)guanidine;
1-(4,6-dimethylpyrimidin-2-yl)-3-(4-(pentyloxy)-3-(trifluoromethyl) phenyl)guanidine;
1-(4-sec-butylphenyl)-3-(4,6-dimethylpyridin-2-yl)guanidine;
1-(4,6-dimethylpyridin-2-yl)-3-(3-phenoxyphenyl)guanidine;
1-(4-butyl-2-methylphenyl)-3-(4,6-dimethylpyridin-2-yl)guanidine;
1-(4-phenyl-benzyl)-3-(4,6-dimethylpyridin-2-yl)guanidine;
1-(4,6-dimethylpyridin-2-yl)-3-(3-phenyl-phenyl)guanidine;
1-(4-(4-chlorophenoxy)phenyl)-3-(4,6-dimethylpyridin-2-yl)guanidine;
1-(4,6-dimethylpyridin-2-yl)-3-(4-(pentyloxy)phenyl)guanidine;
1-(4-bromonaphthalen-1-yl)-3-(4,6-dimethylpyridin-2-yl)guanidine;
1-(3-benzylphenyl)-3-(4,6-dimethylpyridin-2-yl)guanidine;
1-(4-phenoxyphenethyl)-3-(4,6-dimethylpyridin-2-yl)guanidine;
1-(4-butylbenzyl)-3-(4,6-dimethylpyridin-2-yl)guanidine;
1-(4,6-dimethylpyridin-2-yl)-3-(4-pentylphenyl)guanidine;
1-(4-sec-butylphenyl)-3-(6-methylpyridin-2-yl)guanidine;
1-(6-methylpyridin-2-yl)-3-(3-phenoxyphenyl)guanidine;
1-(3-(benzyloxy)phenyl)-3-(6-methylpyridin-2-yl)guanidine;
1-(4-butyl-2-methylphenyl)-3-(6-methylpyridin-2-yl)guanidine;
1-(4-phenylbenzyl)-3-(6-methylpyridin-2-yl)guanidine;
1-(6-methylpyridin-2-yl)-3-(3-phenyl-phenyl)guanidine;
1-(4-(4-chlorophenoxy)phenyl)-3-(6-methylpyridin-2-yl)guanidine;
1-(6-methylpyridin-2-yl)-3-(4-(pentyloxy)phenyl)guanidine;
1-(4-bromonaphthalen-1-yl)-3-(6-methylpyridin-2-yl)guanidine;
1-(3-benzylphenyl)-3-(6-methylpyridin-2-yl)guanidine;
1-(4-phenoxyphenethyl)-3-(6-methylpyridin-2-yl)guanidine;
1-(4-butylbenzyl)-3-(6-methylpyridin-2-yl)guanidine;
1-(6-methylpyridin-2-yl)-3-(4-pentylphenyl)guanidine;
1-(4-sec-butylphenyl)-3-(4-methylpyridin-2-yl)guanidine;
1-(4-ethylphenyl)-3-(4-methylpyridin-2-yl)guanidine;
1-(4-methylpyridin-2-yl)-3-(3-phenoxyphenyl)guanidine;
1-(3-(benzyloxy)phenyl)-3-(4-methylpyridin-2-yl)guanidine;
1-(4-butyl-2-methylphenyl)-3-(4-methylpyridin-2-yl)guanidine;
1-(4-phenyl-benzyl)-3-(4-methylpyridin-2-yl)guanidine;
1-(3-phenyl-phenyl)-3-(4-methylpyridin-2-yl)guanidine;
1-(4-(4-chlorophenoxy)phenyl)-3-(4-methylpyridin-2-yl)guanidine;
1-(4-methylpyridin-2-yl)-3-(4-(pentyloxy)phenyl)guanidine;
1-(4-bromonaphthalen-1-yl)-3-(4-methylpyridin-2-yl)guanidine;
1-(3-benzylphenyl)-3-(4-methylpyridin-2-yl)guanidine;
1-(4-methylpyridin-2-yl)-3-(4-phenoxyphenyl)guanidine;
1-(4-phenoxyphenethyl)-3-(4-methylpyridin-2-yl)guanidine;
1-(4-butylbenzyl)-3-(4-methylpyridin-2-yl)guanidine;
1-(4-methylpyridin-2-yl)-3-(4-pentylphenyl)guanidine;
1-(4-sec-butylphenyl)-3-(4-methylpyrimidin-2-yl)guanidine;
1-(4-phenyl-phenyl)-3-(4-methylpyrimidin-2-yl)guanidine;
1-(4-methylpyrimidin-2-yl)-3-(3-phenoxyphenyl)guanidine;
1-(3-(benzyloxy)phenyl)-3-(4-methylpyrimidin-2-yl)guanidine;
1-(4-butyl-2-methylphenyl)-3-(4-methylpyrimidin-2-yl)guanidine;
1-(4-phenyl-benzyl)-3-(4-methylpyrimidin-2-yl)guanidine;
1-(3-ethylphenyl)-3-(4-methylpyrimidin-2-yl)guanidine;
1-(4-(4-chlorophenoxy)phenyl)-3-(4-methylpyrimidin-2-yl)guanidine;
1-(4-methylpyrimidin-2-yl)-3-(4-(pentyloxy)phenyl)guanidine;
1-(4-bromonaphthalen-1-yl)-3-(4-methylpyrimidin-2-yl)guanidine;
1-(3-benzylphenyl)-3-(4-methylpyrimidin-2-yl)guanidine;
1-(4-methylpyrimidin-2-yl)-3-(4-phenoxyphenyl)guanidine;
1-(4-phenoxyphenethyl)-3-(4-methylpyrimidin-2-yl)guanidine;
1-(4-butylbenzyl)-3-(4-methylpyrimidin-2-yl)guanidine;
1-(4-methylpyrimidin-2-yl)-3-(4-pentylphenyl)guanidine;
1-(4-sec-butylphenyl)-3-(4-methoxy-6-methylpyrimidin-2-yl)guanidine;
1-(4-phenyl-phenyl)-3-(4-methoxy-6-methylpyrimidin-2-yl)guanidine;
1-(4-methoxy-6-methylpyrimidin-2-yl)-3-(3-phenoxyphenyl)guanidine;
1-(3-(benzyloxy)phenyl)-3-(4-methoxy-6-methylpyrimidin-2-yl)guanidine;
1-(4-butyl-2-methylphenyl)-3-(4-methoxy-6-methylpyrimidin-2-yl)guanidine;
1-(4-methoxy-6-methylpyrimidin-2-yl)-3-(3-(methylthio)phenyl)guanidine;
1-(4-phenyl-benzyl)-3-(4-methoxy-6-methylpyrimidin-2-yl)guanidine;
1-(3-phenyl-phenyl)-3-(4-methoxy-6-methylpyrimidin-2-yl)guanidine;
1-(4-(4-chlorophenoxy)phenyl)-3-(4-methoxy-6-methylpyrimidin-2-yl)guanidine;
1-(4-methoxy-6-methylpyrimidin-2-yl)-3-(4-(pentyloxy)phenyl)guanidine;
1-(4-bromonaphthalen-1-yl)-3-(4-methoxy-6-methylpyrimidin-2-yl)guanidine;
1-(3-benzylphenyl)-3-(4-methoxy-6-methylpyrimidin-2-yl)guanidine;
1-(4-phenoxyphenethyl)-3-(4-methoxy-6-methylpyrimidin-2-yl)guanidine;
1-(4-butylbenzyl)-3-(4-methoxy-6-methylpyrimidin-2-yl)guanidine;
1-(4-methoxy-6-methylpyrimidin-2-yl)-3-(4-pentylphenyl)guanidine;
1-(4-sec-butylphenyl)-3-(4,6-dimethoxypyrimidin-2-yl)guanidine;
1-(4,6-dimethoxypyrimidin-2-yl)-3-(4-phenyl-phenyl)guanidine;
1-(4,6-dimethoxypyrimidin-2-yl)-3-(3-phenoxyphenyl)guanidine;
1-(3-(benzyloxy)phenyl)-3-(4,6-dimethoxypyrimidin-2-yl)guanidine;
1-(4-butyl-2-methylphenyl)-3-(4,6-dimethoxypyrimidin-2-yl)guanidine;
1-(4-phenylbenzyl)-3-(4,6-dimethoxypyrimidin-2-yl)guanidine;
1-(4,6-dimethoxypyrimidin-2-yl)-3-(3-phenyl-phenyl)guanidine;
1-(4-(4-chlorophenoxy)phenyl)-3-(4,6-dimethoxypyrimidin-2-yl)guanidine;
1-(4,6-dimethoxypyrimidin-2-yl)-3-(4-(pentyloxy)phenyl)guanidine;
1-(4-bromonaphthalen-1-yl)-3-(4,6-dimethoxypyrimidin-2-yl)guanidine;
1-(3-benzylphenyl)-3-(4,6-dimethoxypyrimidin-2-yl)guanidine;
1-(4,6-dimethoxypyrimidin-2-yl)-3-(4-phenoxyphenyl)guanidine;
1-(4-phenoxyphenethyl)-3-(4,6-dimethoxypyrimidin-2-yl)guanidine;
1-(4-butylbenzyl)-3-(4,6-dimethoxypyrimidin-2-yl)guanidine;
1-(4,6-dimethoxypyrimidin-2-yl)-3-(4-pentylphenyl)guanidine;
1-(4-cyclohexylphenyl)-3-(4,6-dimethylpyridin-2-yl)guanidine;
1-(4-cyclohexylphenyl)-3-(6-methylpyridin-2-yl)guanidine;
1-(4-cyclohexylphenyl)-3-(4-methylpyridin-2-yl)guanidine;
1-(4-cyclohexylphenyl)-3-(4-methylpyrimidin-2-yl)guanidine;
1-(4-cyclohexylphenyl)-3-(4-methoxy-6-methylpyrimidin-2-yl)guanidine;
1-(4-cyclohexylphenyl)-3-(4,6-dimethoxypyrimidin-2-yl)guanidine;
1-(4-cyclohexylphenyl)-3-(4-methylquinazolin-2-yl)guanidine;
1-(4-sec-butylphenyl)-3-(4-methylquinazolin-2-yl)guanidine;
1-(4-phenyl-phenyl)-3-(4-methylquinazolin-2-yl)guanidine;
1-(4-methylquinazolin-2-yl)-3-(3-phenoxyphenyl)guanidine;
1-(3-(benzyloxy)phenyl)-3-(4-methylquinazolin-2-yl)guanidine;
1-(4-butyl-2-methylphenyl)-3-(4-methylquinazolin-2-yl)guanidine;
1-(4-phenyl-benzyl)-3-(4-methylquinazolin-2-yl)guanidine;
1-(3-phenyl-phenyl)-3-(4-methylquinazolin-2-yl)guanidine;
1-(4-(4-chlorophenoxy)phenyl)-3-(4-methylquinazolin-2-yl)guanidine;
1-(4-methylquinazolin-2-yl)-3-(4-(pentyloxy)phenyl)guanidine;
1-(4-bromonaphthalen-1-yl)-3-(4-methylquinazolin-2-yl)guanidine;
1-(3-benzylphenyl)-3-(4-methylquinazolin-2-yl)guanidine;
1-(4-methylquinazolin-2-yl)-3-(4-phenoxyphenyl)guanidine;
1-(4-phenoxyphenethyl)-3-(4-methylquinazolin-2-yl)guanidine;
1-(4-butylbenzyl)-3-(4-methylquinazolin-2-yl)guanidine;
1-(4-methylquinazolin-2-yl)-3-(4-pentylphenyl)guanidine;
1-(4-sec-butylphenyl)-3-(4-methyl-5,6,7,8-tetrahydroquinazolin-2-yl)guanidine;
1-(4-phenyl-phenyl)-3-(4-methyl-5,6,7,8-tetrahydroquinazolin-2-yl)guanidine;
1-(4-methyl-5,6,7,8-tetrahydroquinazolin-2-yl)-3-(3-phenoxyphenyl)guanidine;
1-(3-(benzyloxy)phenyl)-3-(4-methyl-5,6,7,8-tetrahydroquinazolin-2-yl)guanidine;
1-(4-butyl-2-methylphenyl)-3-(4-methyl-5,6,7,8-tetrahydroquinazolin-2-yl)guanidine;
1-(4-phenyl-benzyl)-3-(4-methyl-5,6,7,8-tetrahydroquinazolin-2-yl)guanidine;
1-(3-phenyl-phenyl)-3-(4-methyl-5,6,7,8-tetrahydroquinazolin-2-yl)guanidine;
1-(4-(4-chlorophenoxy)phenyl)-3-(4-methyl-5,6,7,8-tetrahydroquinazolin-2-yl)guanidine;
1-(4-methyl-5,6,7,8-tetrahydroquinazolin-2-yl)-3-(4-(pentyloxy)phenyl)guanidine;
1-(3-benzylphenyl)-3-(4-methyl-5,6,7,8-tetrahydroquinazolin-2-yl)guanidine;
1-(4-phenoxyphenethyl)-3-(4-methyl-5,6,7,8-tetrahydroquinazolin-2-yl)guanidine;
1-(4-butylbenzyl)-3-(4-methyl-5,6,7,8-tetrahydroquinazolin-2-yl)guanidine;
1-(4-methyl-5,6,7,8-tetrahydroquinazolin-2-yl)-3-(4-pentylphenyl)guanidine;
1-(3-benzylphenyl)-3-m-tolylguanidine;
1-(3-(pentyloxy)phenyl)-3-m-tolylguanidine;
1-(3-phenoxyphenyl)-3-m-tolylguanidine;
1-(4-tert-butylbenzyl)-3-(4,6-dimethylpyridin-2-yl)guanidine;
1-(4-tert-butylbenzyl)-3-(6-methylpyridin-2-yl)guanidine;
1-(4-tert-butylbenzyl)-3-(4-methylquinazolin-2-yl)guanidine;
1-nicotinoyl-3-(3-phenoxyphenyl)guanidine;
1,1-dinicotinoyl-3-(3-phenoxyphenyl) guanidine;
1-nicotinoyl-3-(4-(pentyloxy)phenyl) guanidine; or
1,1-dinicotinoyl-3-(4-(pentyloxy) phenyl)guanidine.

48. (canceled)

49. A pharmaceutical composition comprising a compound or salt of the formula 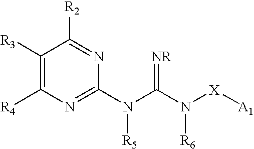
or a pharmaceutically acceptable salt thereof, wherein:
X is absent, —CR′R″—, —(CR′R″)₂—, —CR′R″O—, —O—, or NR;
Each R is independently hydrogen, C₁-C₆alkyl, or cyano;
R′ and R″ are independently hydrogen, halogen, C₁-C₂alkyl, or C₁-C₂alkoxy;
R₂ is C₁-C₂alkyl or C₁-C₂alkoxy;
R₃ and R₄ are independently hydrogen, halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy); or
R₃ and R₄ are joined to form a 5- or 6-membered carbocyclic ring, or a 5- or 6-membered heterocyclic ring containing 1 or 2 N, O, or S heteroatoms; each of which carbocyclic or heterocyclic ring is substituted with 0, or 1 or more substituents chosen from halogen, hydroxy, cyano, acetyl, C₁-C₄alkyl, C₁-C₄alkoxy, C₁-C₂haloalkyl, C₁-C₂haloalkoxy, C₁-C₄alkylthio, and mono- and di-(C₁-C₄alkyl)amino;
R₅ and R₆ are independently hydrogen, C₁-C₄alkyl, C₂-C₄alkenyl, or C₃-C₈cycloalkyl(C₀-C₂alkyl);
A₁ is
i) phenyl, which is substituted with 1 LA₂ substituent and 0 or 1 or more R₇ substituents; or
ii) naphthyl, indanyl, or 9H-fluorenyl, each of which is substituted with 0 or 1 LA₂ substituent, and 0 or 1 or more R₇ substituents; or
iii) heteroaryl, which is substituted with 0 or 1 LA₂ substituent, and 0 or 1 or more R₇ substituents;
R₇ is independently halogen, hydroxy, cyano, nitro, amino, acetyl, oxo, —SH, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, mono- or di-(C₁-C₆alkyl)amino, mono- or di-(C₁-C₄alkyl)amino(C₁-C₄alkyl), or mono- or di-(C₁-C₄alkyl)amino(C₁-C₄alkoxy);
L is absent, —CR₈R₉—, —(CR₈R₉)₂—, —CR₈R₉O—, —OCR₈R₉—, —O(CR₈R₉)₂—, —NH(C═O)—, or —O—, where R₈ and R₉ are independently hydrogen, halogen, C₁-C₂alkyl, or C₁-C₂alkoxy; and
A₂ is C₃-C₆alkyl, C₃-C₆alkenyl, C₃-C₆alkynyl, a carbocyclic group, or a heterocyclic group, each of which is substituted with 0, 1 or more substituents independently chosen from halogen, hydroxy, cyano, nitro, amino, acetyl, oxo, C₁-C₆alkyl, C₂-C₆alkenyl, C₂-C₆alkynyl, C₁-C₆alkoxy, C₁-C₄alkoxy(C₁-C₄alkyl), C₁-C₄alkoxy(C₁-C₄alkoxy), C₁-C₄alkoxy(C₁-C₄alkylamino), C₁-C₆hydroxyalkyl, C₁-C₆aminoalkyl, C₃-C₈cycloalkyl(C₀-C₂alkyl), C₁-C₂haloalkyl, C₁-C₂haloalkoxy, C₂-C₆alkanoyl, C₁-C₄alkylthio, and mono- and di-(C₁-C₆alkyl)amino, with the proviso that when R₂ and R₄ are both methyl and R₃ is hydrogen.

50. A pharmaceutical composition of claim 49 or 50, wherein the composition is formulated as an injectable fluid, an aerosol, a cream, a gel, a tablet, a pill, a capsule, a syrup, ophthalmic solution, or a transdermal patch.

51. A package comprising a pharmaceutical composition of claim 50 in a container and further comprising instructions for using the composition to treat a patient suffering from Hepatitis C infection.

52-53. (canceled)

54. A method for treating Hepatitis C infection comprising administering to a patient in need of such treatment a therapeutically effective amount of a compound or salt of claim 1.

55-57. (canceled)

58. A method for treating Hepatitis C infection comprising administering to a patient in need of such treatment a therapeutically effective amount of a compound or salt of claim 3.
